(19) United States
(12) Patent Application Publication
(10) Pub. No.: US 2011/0105855 A1
(43) Pub. Date: May 5, 2011

(54) EARLY DETECTION OF COGNITION IMPAIRMENT IN ELDERLY POPULATION

(75) Inventor: ELIZABETH J. JOHNSON, West Roxbury, MA (US)

(73) Assignee: TRUSTEES OF TUFTS COLLEGE, Medford, MA (US)

(21) Appl. No.: 12/907,422

(22) Filed: Oct. 19, 2010

Related U.S. Application Data
(60) Provisional application No. 61/256,449, filed on Oct. 30, 2009.

(51) Int. Cl. A61B 5/00; C12Q 1/02

(52) U.S. Cl. 600/300; 435/29

(57) ABSTRACT

We have discovered novel methods and systems for detecting cognitive impairment in a subject by analyzing the level of xantophylls in macula lutea or red blood cell sample from the subject. We have discovered that decrease of xanthopyll level in the subject is indicative of cognitive impairment.

### CROSS-REFERENCE TO RELATED APPLICATIONS

This application claims benefit under 35 U.S.C. §119(e) of provisional application No. 61/256,449, filed on Oct. 30, 2009, the content of which is incorporated herein by reference in its entirety.

### GOVERNMENT SUPPORT

This invention was made with government support under contract No. USDA 58-1950-7-707 awarded by the United States Department of Agriculture. The government has certain rights in the invention.

### BACKGROUND OF INVENTION

Mild cognitive impairment (MCI) is prevalent in the elderly human population. According to the American College of Physicians, mild cognitive impairment affects about 20 percent of the population over 70 and it is estimated that up to one third of adults will experience a gradual decline in cognitive function known as MCI as they age.

MCI is defined as cognitive defects that do not interfere with daily living. It may include slower thinking, a reduced ability to learn, and impaired memory. This condition may encompass deficiencies in any or all of the following categories: Language—words don't come as quickly as they once did; Visuospatial ability—placement of things in time and space becomes more difficult, such as having trouble getting the proportions right when drawing a box; Executive function—decision making becomes more challenging; Memory—recent recall diminishes, such as what one did yesterday. Senior individuals with MCI are able to function in everyday activities and otherwise function normally in society but their thinking abilities are mildly impaired, for example, they would have difficulty with memory—trouble remembering the names of people they met recently, remembering the flow of a conversation, and a tendency to misplace things. The individual may be aware of these difficulties and compensate with increased reliance on notes and calendars. As such, the symptoms of mild cognitive impairment can be so subtle that they are difficult to detect unless living with the affected person.

Most but not all patients with MCI develop a progressive decline in their thinking abilities over time, and Alzheimer's disease is usually the underlying cause. Mayo Clinic researchers have found that MCI is considered a strong early predictor of Alzheimer's disease and is prevalent among the elderly and increases with age and fewer years of education.

Since detection and diagnosis of MCI in the elderly is often difficult because it is confused as a normal phase of aging and/or the patient compensates with some coping strategies such as the use of notes and calendars, the development of biomarkers for the decline in cognitive function in elderly people can provide methods of early detection. Such early detection methods can be implemented as part of the routine annual physical of a subject 65 years or older. Early detection of MCI provides better management of MCI and the underlying causes, and better planning for the future in elderly people.

### SUMMARY OF THE INVENTION

Embodiments of the present invention are based on the discovery that the lutein (L) and zeaxanthin (Z) levels in the macula lutea positively correlate with those in the occipital cortex. The L and Z levels extracted from the membranes of red blood cells (RBCs) also positively correlate with those in the macula lutea. These correlations provide an accurate and non-invasive method for determining the level of xanthophylls in the brain as oppose to a brain tissue biopsy every time the levels of xanthophyll in the brain need to be evaluated. Measurements of the L plus Z levels in the macula lutea as macular pigment optical density (MPOD) units or measurements of the RBCs L and/or Z levels give representative values of L and/or Z in the brain. Reports have shown that among the elderly population, subjects with MCI tended to have lower plasma xanthophyll levels. Since xanthophylls are known to protect neuron from apoptosis, by using the levels of xanthophylls as an indicator/biomarker of MCI, an accurate and non-invasive method for determining the levels of xanthophyll in the brain can allow a physician to detect early MC, detect risk of onset of MCI, and monitor the efficacy and dosage of dietary xanthophyll supplements.

Accordingly, provided herein is a method of diagnosing of MCI in a subject comprising measuring a xanthophyll level in macula lutea or in a RBC sample from the subject and comparing the xanthophyll level with a reference xanthophyll level, wherein if the measured xanthophyll level from the subject is lower than the reference xanthophyll level the subject is affected with MCI. The xanthophyll level is lower by at least 10% than the reference xanthophyll level, at least 20%, at least 30%, at least 40%, at least 50%, at least 60%, at least 70%, at least 80%, at least 90%, 100%, 200%, 300% or 1000%, including all the percentages between 10-1000%.

Embodiments of the present invention also provide a method of determining a need for a dietary supplementation of xanthophylls in a subject, the method comprising: (a) measuring a xanthophyll level in a macula lutea or in a RBC sample from a subject at a first time point; (b) measuring a xanthophyll level in the subject at a second time point using the same method as in step (a), wherein the xanthophyll measured in step (a) and (b) are the same; (c) comparing the xanthophyll level obtained at the first time point with that obtained at the second time point, wherein if there is a decrease in the level of xanthophyll at the second time point compared to the first time point, the subject is in need of dietary supplementation of xanthophylls. The xanthophyll level is considered decreased if the decrease is compared to the xanthophyll level at the first time point by at least 10%, at least 20%, at least 30%, at least 40%, at least 50%, at least 60%, at least 70%, at least 80%, at least 90%, 100%, 200%, 300% or 1000%, including all the percentages between 10-1000%.

In one embodiment, the first and second time points are separated by about 3-6 months. In another embodiment, they are separated by about one year.

In one embodiment, the method further comprises measuring a level of xanthophyll at a third time point and comparing the level of xanthophyll obtained at the third time point with that obtained at the first and/or second time point, wherein the second and third time points are separated by 1-6 months, wherein if there is a decrease in the level of xanthophyll at the third time point compared to the first and/or second time point, the subject is in need of dietary supplementation of xanthophylls.

In another embodiment, the present invention provides a method of monitoring the amount of dietary supplementation with xanthophylls in a subject taking xanthophyll supplements, the method comprising (a) measuring a xanthophyll level in a macula lutea or in a RBC sample from a subject at a first time point; (b) measuring a xanthophyll level in the subject at a second time point using the same method as in step (a), wherein the xanthophyll measured in step (a) and (b) are the same; (c) comparing the xanthophyll level obtained at the first time point with that obtained at the second time point, wherein if there is a decrease in the level of xanthophyll at the second time point compared to the first time point, the subject is in need of increase in the dietary supplementation of xanthophylls.

In some aspects, the xanthophyll measured in any of the methods or systems described can be L, a combination of L and Z, or Z.

In some aspects, the measuring in any of the methods or systems described herein is performed by measuring the macular pigment optical density (MPOD) of the macula lutea using light absorption of xanthophylls in a densitometer, and the wavelength of the absorbed light is between 450-495 nm. In some aspects, the measuring in any of the methods or systems described is performed using heterchromatic flicker photometry (HFP).

In some aspects any of the methods or systems described herein, the xanthophyll level is measured from RBCs and the xanthophyll is extracted from the RBCs with an organic solvent. In some aspects, the organic solvent is selected from a group consisting of chloroform, methanol and hexane. In some aspects, the level of the extracted xanthophylls is measured by high performance liquid chromatography (HPLC).

In one embodiment of any of the methods or systems described herein, the subject is 65 years old or older. In another embodiment of any of the methods or systems described herein, the subject is 40 years old or older and has a family history of medical conditions that can cause MCI such as Alzheimer's disease.

In one embodiment of any of the methods or systems described herein, the subject is not currently affected with cancer, Alzheimer's disease, dementia, Creutzfeldt—Jakob disease, abnormal thyroid stimulating hormone, vitamin B12 deficiency, syphilis, sleep apnea, or Parkinson disease.

In one embodiment of any of the methods or systems described herein, the reference xanthophyll level is an average level obtained from a population of healthy subjects without symptoms associated with MCI and is measured by the same method as that used for measuring the xanthophyll level in the subject. Preferably the reference level is measured from subjects of similar age and of the same gender as the subject in need of diagnosis.

In one embodiment of any of the methods or systems described herein, the subject is at risk of developing MCI, such as the subject has a vascular disease, is obese, has a high stress job (e.g. air traffic controller, stock market floor trader), is diabetic and/or and has a family history of medical conditions that can cause MCI such as Alzheimer's disease.

In another embodiment, provided herein is a system comprising: (a) a measuring module quantifying a xanthophyll level comprising a signal from an absorption of light having a wavelength between 450-495 nm capable of indicating a level of xanthophyll in a subject; (b) a storage module configured to store data output from the measuring module; (c) a comparison module adapted to compare the data stored on the storage module with a reference and/or control data, and to provide a retrieved content, and (d) an output module for displaying the retrieved content for the user, wherein the retrieved content the level of xanthophyll lower than the reference and/or control data indicates that the subject is affected with MCI.

In one embodiment, the reference and/or control data comprises data from a population of a population of healthy subjects who do not have symptoms associated with MCI.

In another embodiment, provided herein is a system to facilitate the diagnosis of MCI and/or monitoring development of MCI in a subject, comprising: (a) a determination module configured to receive and output a xanthophyll level obtained from a subject, wherein the xanthophyll level is measured by an absorption of light with a wavelength between 450-495 nm; (b) a storage module configured to store output data from the determination module; (c) a comparison module adapted to compare the output data stored on the storage module with a reference and/or control data, and to provide a comparison content, and (d) an output module for displaying the comparison content for the user, wherein if the measured level of xanthophyll from the subject is lower than the reference and/or control data indicates that the subject affected with MCI or if there is a reduction of at least 10% to a prior reading, then the subject likely has developed MCI and is advised to take xanthophyll dietary supplement.

In one embodiment, the reference and/or control data comprises previous data from the same subject.

In another embodiment, provided herein is a computer readable storage medium comprising: (a) a storing data module containing data from a subject that represents as a signal from the absorption of light with wavelength between 450-495 nm indicating a level of xanthophyll; (b) a comparison module that compares the data stored on the storing data module with a reference data and/or control data, and to provide a comparison content, and (c) an output module displaying the comparison content for the user, wherein if the measured level of xanthophyll from the subject is lower than a reference level of xanthophyll indicates that the subject affected with MCI or if there is a reduction of at least 10% to a prior reading, then the subject likely has developed MCI and is advised to take xanthophyll dietary supplement.

In one aspect, the reference and/or control data of this system comprises data from a plurality of subjects who do not have symptoms associated with MCI.

### BRIEF DESCRIPTION OF THE DRAWINGS

FIGS. 1A-1G show data regarding lutein (L) and zeaxanthin (Z) levels in a primate (monkey) models. FIG. 1A shows the positive correlation of the levels of lutein (L) present in the primate retina and in the occipital cortex (n=4). FIG. 1B shows the positive correlation of the levels of zeaxanthin (Z) present in the primate retina and in the occipital cortex (n=4). FIG. 1C shows a positive correlation of the levels of L present in the primate retina and in the cerebellum (n=6). FIG. 1D shows a positive correlation of the levels of L present in the primate retina and in the occipital cortex (n=6). FIG. 1E shows, a positive correlation of the levels of Z present in the primate retina and in the cerebellum (n=5).

FIG. 1F shows the positive correlation of the levels of Z present in the primate retina and in the occipital cortex (n=5). FIG. 1G shows the positive correlation of the levels of Z present in the primate retina and in the occipital cortex (n=6). Number of monkeys studied=n.

FIG. 2 is a block diagram showing an exemplary system for the diagnosis of mild cognition impairment (MCI) and/or monitoring of cognition performance.

FIG. 3 is an exemplary set of instructions on a computer readable storage medium for use with the systems described herein.

FIG. 4 shows a correlation of mild cognitive impairment, as assessed by the Mini Mental State Exam Score (MMSE), with Macular Pigment Optical Density (MPOD) in older adults (n=99). (Controlling for BMI, age, smoking status, gender and ethnicity).

FIG. 5 shows that the combined RBC membrane lutein (L) and zeaxanthin (Z) level and also the combined plasma L and Z level correlate positively with Macular Pigment Optical Density (MPOD) in healthy adults, with the RBC membrane being a better incriminator of small changes in MPOD compared to plasma.

### DETAILED DESCRIPTION OF THE INVENTION

The invention is generally related to methods and systems for assisting in diagnosis and follow-up of cognitive function in a human subject. Specifically, we have discovered that lower than average normal level of xanthophylls, particularly as measured either from macula lutea or from red blood cells of a human subject is indicative of cognitive impairment, even when the cognitive impairment is mild.

Unless otherwise explained, all technical and scientific terms used herein have the same meaning as commonly understood by one of ordinary skill in the art to which this disclosure belongs. Definitions of common terms in diseases and disorders, separation and detection techniques can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise stated, the present invention is performed using standard procedures, as described, for example in Modern HPLC for Practicing Scientists by Michael W. Dong, Wiley-Interscience; 1st edition (2006); Practical HPLC Method Development by Lloyd R. Snyder, Wiley-Interscience; 2nd edition (1997); Analysis of lipids by Kumar Deb Mukherjee, Nikolaus Weber (1993), which are all incorporated by reference herein in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and the include plural referents unless context clearly indicates otherwise. Similarly, the word or is intended to include and unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified throughout the specification are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### DEFINITIONS OF TERMS

The term “subject” includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In one embodiment, the subject is a mammal. In one embodiment, the subject is a human subject.

As used herein, the term “comprising” means that other elements can also be present in addition to the defined elements presented. The use of “comprising” indicates inclusion rather than limitation.

The term “consisting of” refers to the components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention

As used herein, the terms “macula lutea”, “macula” and “yellow spot” refer to the region on the retina where L and Z are highly concentrated. The terms “macula lutea”, “macula” and “yellow spot” are used interchangeably.

As used herein, the term “xanthophyll” refers to L and/or Z xanthophyll.

As used herein, the term “neuroprotective” refers to protection of the nervous system by the prevention of neurons from apoptosis or degeneration, for example following a brain injury, natural aging or as a result of chronic neurodegenerative diseases such as Alzheimer's disease and Parkinson's disease.

Numerous studies indicate that carotenoids function with as anti-oxidants and have protective properties towards the cells of the body. For example, carotenoids have been reported to reduce the risk of developing cancer and/or cancer recurrence. Increase levels of carotenoids indicate greater likelihood of recurrence-free survival in women who have been diagnosed with early-stage breast cancer. In the brain, lower levels of carotenoids have been shown to associate with reduced cognition performance and function in elderly population. Dietary lutein supplement in older women improved verbal fluency, memory and rate of learning. Surveillance of the level of carotenoids in the brain can provide an indication of onset of reduced cognition performance and function in an elderly population, and/or provide an indication of a need to raise the level of carotenoids to levels that is considered neuroprotective.

Although carotenoids are importance, most animals cannot synthesize carotenoids and require dietary intake of carotenoids in the form of vegetables and fruits. The ingested carotenoids are then distributed to throughout body. The common method of determining carotenoid concentration in the body is by measurement of the carotenoid content in the plasma. However, plasma carotenoid concentration does not accurately reflect the long term concentration of carotenoids in the body, providing only a “snapshot” picture of the xanthophyll levels in an individual. Therefore it cannot be used to estimate the levels of xanthophylls accumulated. We have discovered that the accumulation of xanthophylls in the macula lutea provides an accurate correlation to the amount of xanthophylls in the brain. Surprisingly, we found this phenomenon to be true also in the RBCs that circulate for about four months in an individual. Thus measurement of the xanthophyll levels in the macula and/or circulating RBCs provide much better estimates of xanthophylls accumulation than plasma.

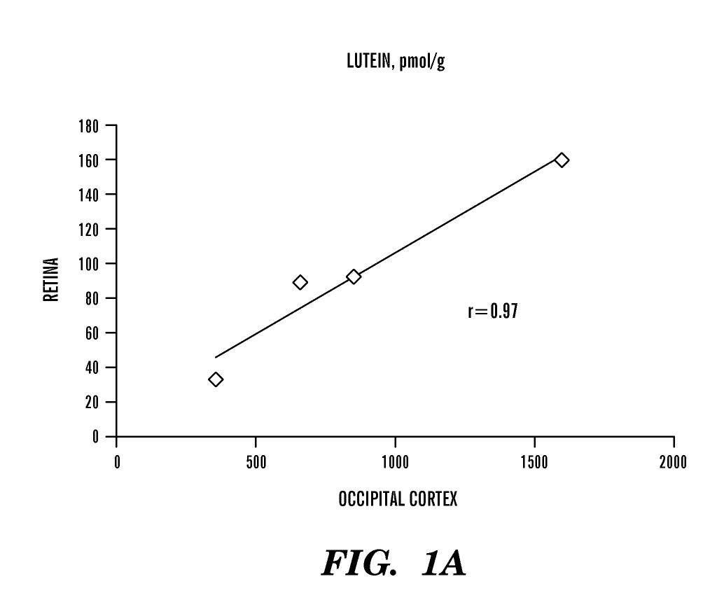

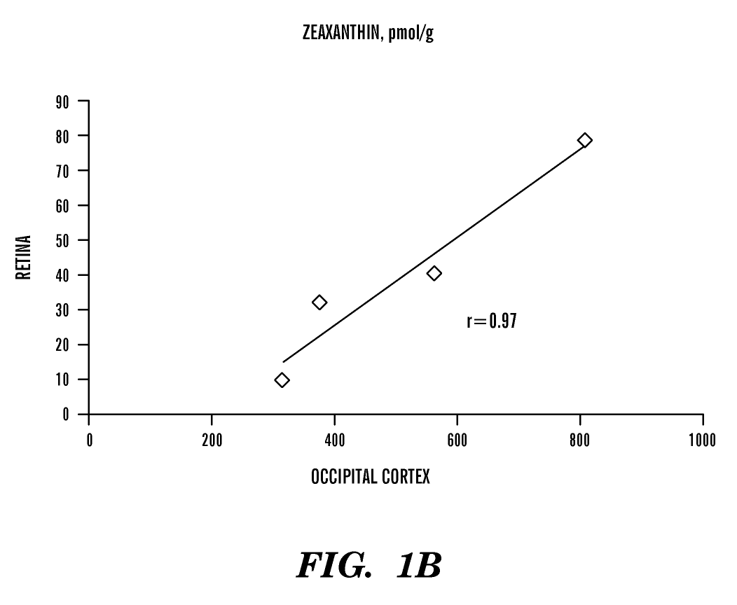

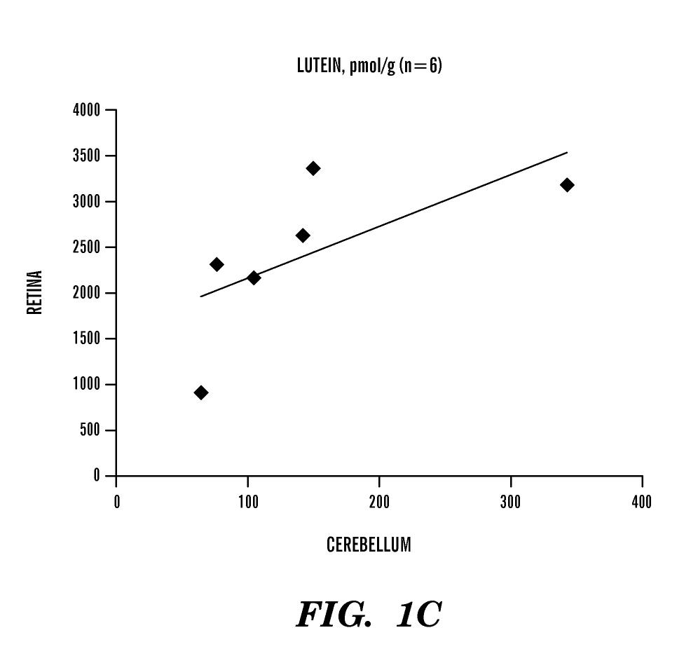

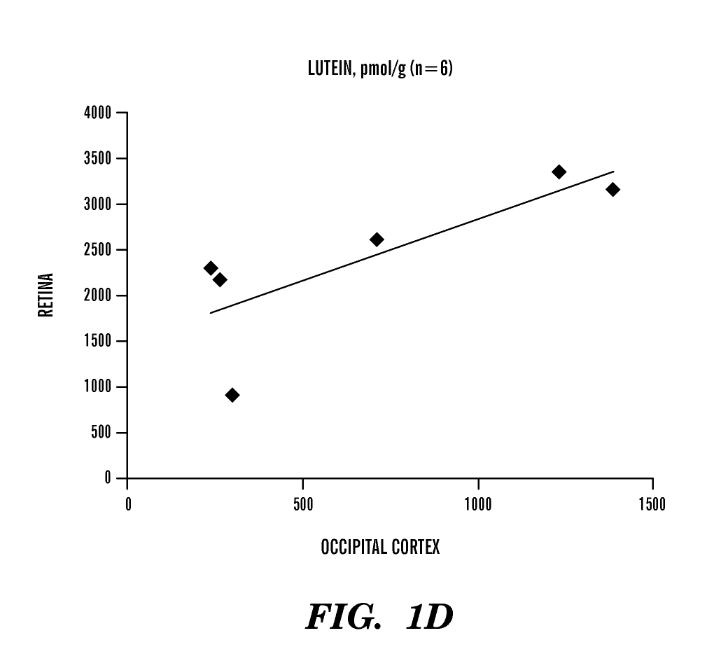

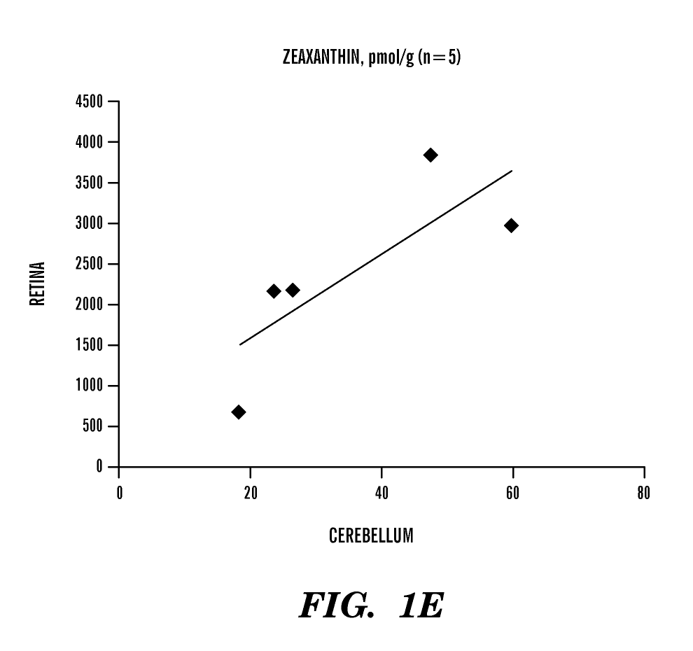

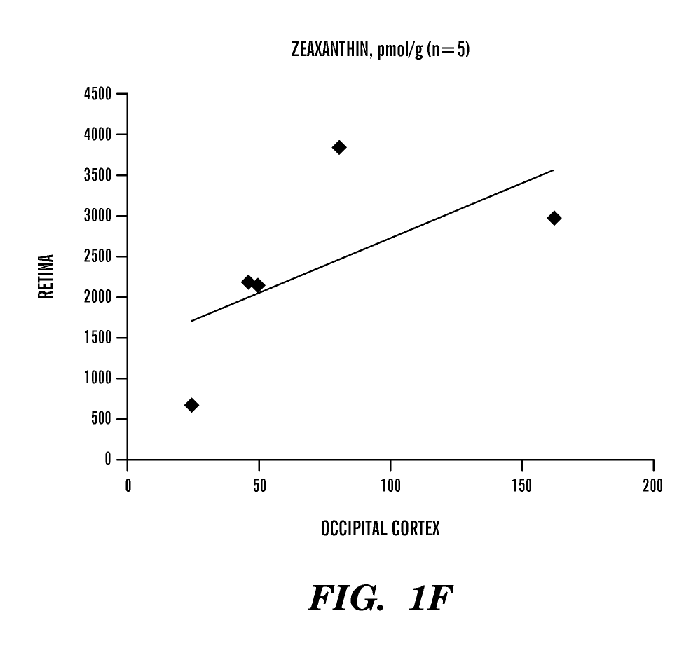

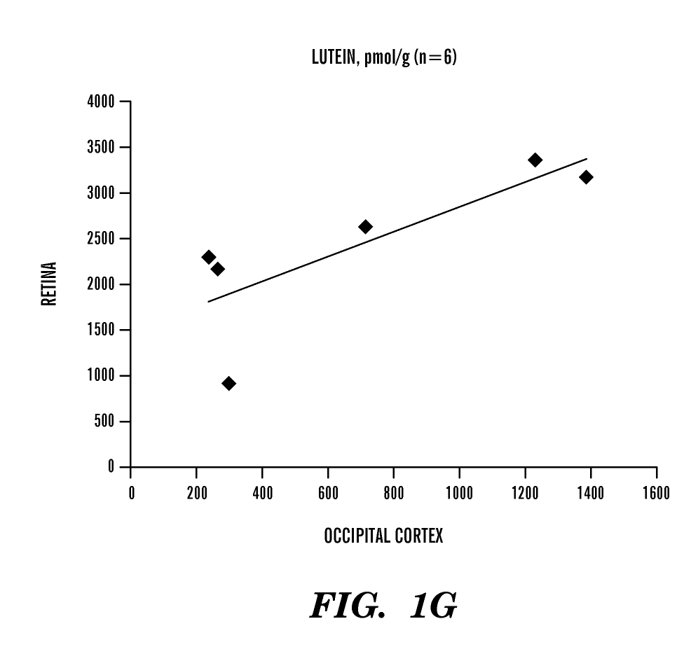

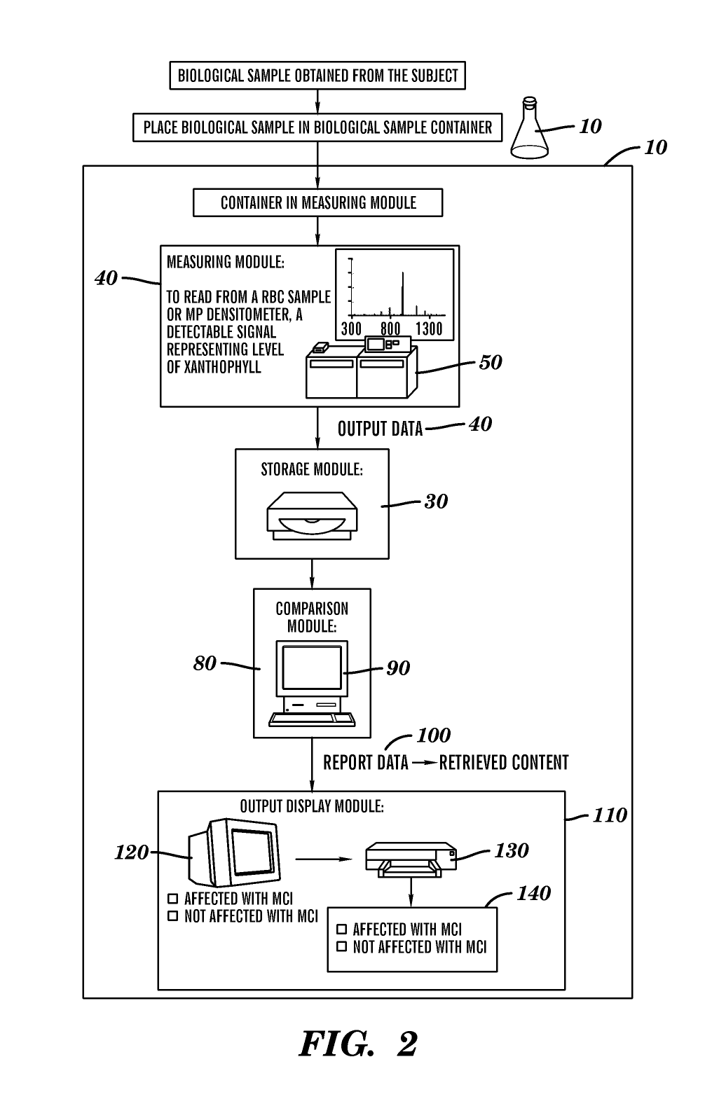

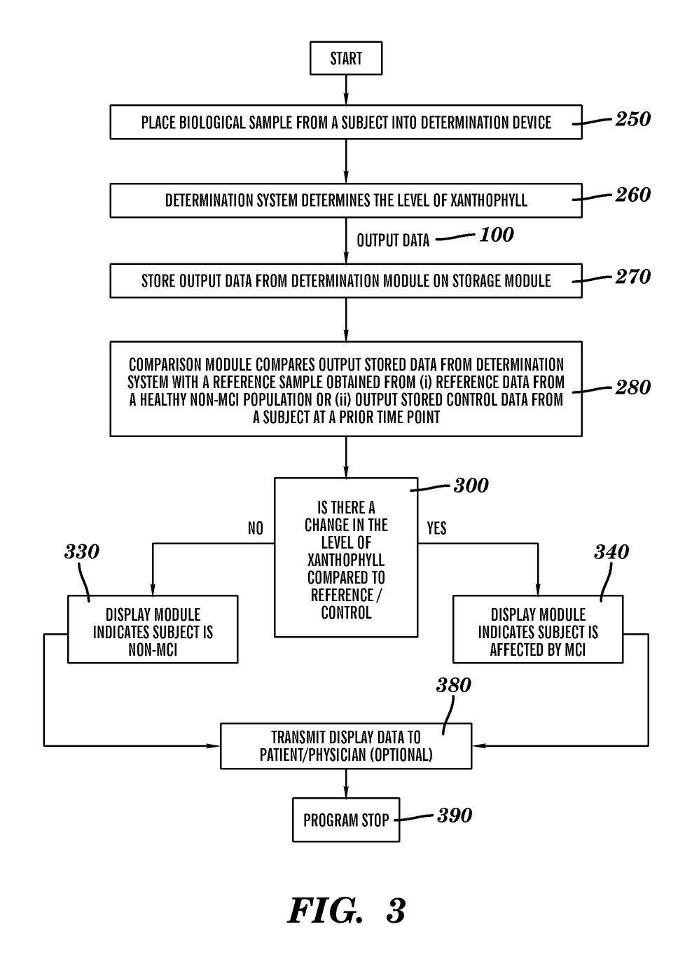

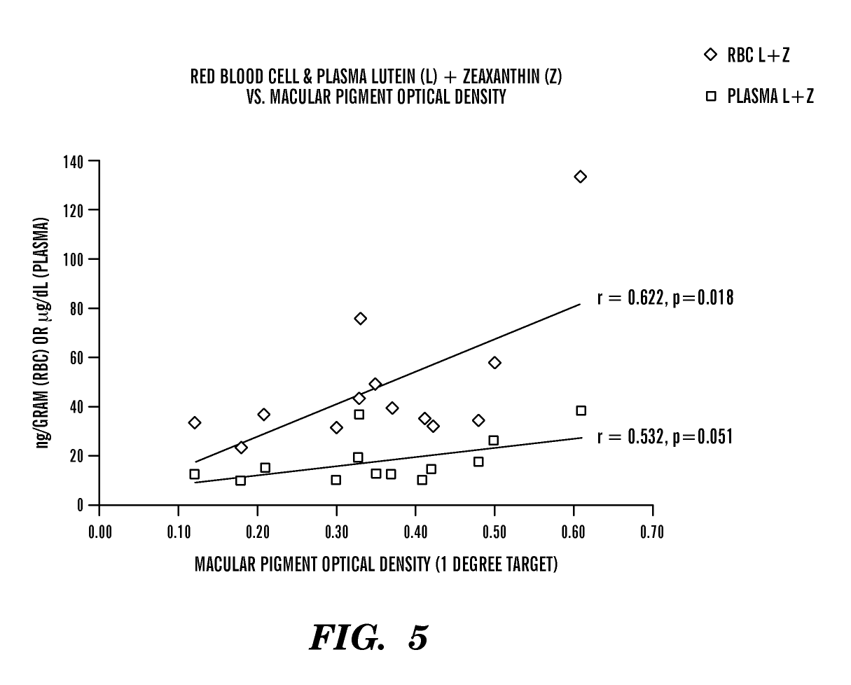

The inventors have found that the levels of xanthophylls, in particular, L and Z, in the primate retina positively correlates with the level of xanthophylls present in the occipital cortex (FIGS. 1B, 1D and 1E showing r-values ranging between 0.623 and 0.796). The L and Z levels extracted from the membranes of red blood cells (RBCs) also positively correlate with those in the macula lutea. These correlations provide an accurate and non-invasive method for determining the level of xanthophylls in the brain as oppose to a brain tissue biopsy every time the levels of xanthophyll in the brain need to be evaluated. Measurements of the L plus Z levels in the macula lutea as macular pigment optical density (MPOD) units or measurements of the RBC L and/or Z levels give representative values of L and/or Z in the brain. Both plasma and RBC concentrations of xanthophylls were significantly related to MPOD; however the strength of the relationship was stronger for RBC (FIG. 5). RBC measures of xanthophyll have a greater correlation with MPOD than measures in plasma. This is due to plasma reflecting short-term intakes and RBC reflecting long-term intakes. The steeper slope indicates that RBC xanthophyll is more sensitive to fluctuations in MPOD, and therefore would be a better indicator of the levels of xanthophylls in the macula and in the brain. Major carotenoids identified in the human brain are lutein, zeaxanthin, anhydrolutein, alpha-cryptoxanthin, beta-cryptoxanthin, alpha-carotene, cis- and transbetacarotene, and cis- and trans-lycopene. Xanthophylls (oxygenated carotenoids) accounted for 66-77% of total carotenoids in all brain regions examined. Such observed correlation is useful for a more accurate determination of the level of xanthophylls in the brain of a subject.

Accordingly, an embodiment of the present invention provides a method of diagnosing of cognitive impairment, such as mild cognitive impairment (MCI) in a subject comprising measuring a xanthophyll level in macula lutea or in a red blood cell (RBC) sample from the subject and comparing the xanthophyll level with a reference xanthophyll level, wherein if the measured xanthophyll level from the subject is lower than the reference xanthophyll level the subject is affected with MCI. The xanthophyll level from the subject is lower by at least 10%, at least 20%, at least 30%, at least 40%, at least 50%, at least 60%, at least 70%, at least 80%, at least 90%, 100%, 200%, 300% or 1000%, including all the percentages between 10-1000%. In one embodiment, a lower xanthophyll level compared to the reference indicates a likelihood of MCI and the subject is encourage to have a comprehensive physical to rule some common causes of MCI-like symptoms such as low levels of thyroid hormone, vitamin B12 deficiency, depression, anxiety, syphilis, and sleep apnea etc. The subject is also encouraged to undergo cognition testing. The goal is to use the xanthophyll level in the brain for early diagnose MCI and/or detect low or poor dietary intake of xanthophylls.

The invention also provides an assay to measure cognitive impairment. The assay comprises or consists essentially of a system for measuring xanthophyll level from a red blood cell sample, and a system for comparing the amount in the test sample to a reference value. The reference value can be obtained either from a control sample, which may be a red blood sample from an individual without cognitive impairment, a pooled sample from such individuals or a reference value or number previously obtained using such samples to give a reference value or a range of normal reference values. If the comparison system, which may be a computer implemented system, indicates that the red blood cell xanthophyll level is below the reference value, the subject from whom the sample is derived can be diagnosed as having at least a mild cognitive impairment. Such subject may then be further administered agents that are indicated fro increasing xanthophyll levels to assist in slowing down or reversing the mild cognitive impairment.

Another embodiment of the present invention is a method of determining a need for a dietary supplementation of xanthophylls in a subject, the method comprising: (a) measuring a xanthophyll level in a macula lutea or in a RBC sample from a subject at a first time point; (b) measuring a xanthophyll level in the subject at a second time point using the same method as in step (a), wherein the xanthophyll measured in step (a) and (b) are the same; and (c) comparing the xanthophyll level obtained at the first time point with that obtained at the second time point, wherein if there is a decrease in the level of xanthophyll at the second time point compared to the first time point, the subject is in need of dietary supplementation of xanthophylls. The decrease in the level of xanthophyll at the second time point compared to the first time point is at least 5%, 10%, at least 20%, at least 30%, at least 40%, at least 50%, at least 60%, at least 70%, at least 80%, at least 90%, 100%, 200%, 300% or 1000%, including all the percentages between 10-1000%.

In one embodiment, the first and second time points are separated by about 3-6 months.

In another embodiment, the first and second time points are separated by about one year.

The invention naturally contemplates measuring more than first and second time points, such, that the method can be part of, e.g., a yearly or bi-yearly or quarterly follow-up screen for individuals, such as individuals at risk of developing cognitive impairment.

In another embodiment, the invention provides a method for determining a need for a dietary supplementation of xanthophylls in a subject described herein further comprising measuring a level of xanthophyll at a third time point and comparing the level of xanthophyll obtained at the third time point with that obtained at the first and/or second time point, wherein the second and third time points are separated by 1-6 months, wherein if there is a decrease in the level of xanthophyll at the third time point compared to the first and/or second time point, the subject is in need of dietary supplementation of xanthophylls.

Another embodiment of the present invention is method of monitoring the amount of dietary supplementation with xanthophylls in a subject taking xanthophyll supplements, the method comprising (a) measuring a xanthophyll level in a macula lutea or in a RBC sample from a subject at a first time point; (b) measuring a xanthophyll level in the subject at a second time point using the same method as in step (a), wherein the xanthophyll measured in step (a) and (b) are the same; (c) comparing the xanthophyll level obtained at the first time point with that obtained at the second time point, wherein if there is a decrease in the level of xanthophyll at the second time point compared to the first time point, the subject is in need of increase in the dietary supplementation of xanthophylls. The decrease in the level of xanthophyll at the second time point compared to the first time point is at least 10%, at least 20%, at least 30%, at least 40%, at least 50%, at least 60%, at least 70%, at least 80%, at least 90%, 100%, 200%, 300% or 1000%, including all the percentages between 10-1000%.

By monitoring the xanthophyll level regularly over time, for example, once a month, once every two months or even once every six months or a year or two years, etc, plotting the xanthophyll level versus time, and comparing with prior xanthophyll levels, it is possible to detect any fluctuations of said levels in the brain. For example, the method described herein allow evaluation of whether the xanthophyll levels in the brain of a subject is holding steady at the level that is considered to be neuroprotective, whether the xanthophyll level is or has fallen to below the level that is considered to be neuroprotective, or whether the dietary supplement intake has helped in increasing the xanthophyll level to a level that is considered to be neuroprotective compared to before the subject went on the dietary supplement of xanthophylls. As an exemplary, in surveillance of dietary supplement requirement and adjustments, using the method described herein, a subject has been found to have about 25% lower amount of L and Z in the brain compare the average healthy population. This subject then starts a regiment of L and Z daily supplements, at 5 mg/day of L and 1 mg/day of Z. After two months, the same method is used again to evaluate whether the prescribed dosages have been sufficient to increase the xanthophyll in the brain to the xanthophyll level observed in the average healthy population. The xanthophyll level in the subject has increased to just about 5% lower amount of L and Z in the brain compare the average healthy population, indicating that the dosage is insufficient for this subject. The dosage of L and Z is then adjusted to at 7 mg/day of L and 2 mg/day of Z. After another two months, the xanthophyll level is tested again and has increased to about 2% higher amount of L and Z in the brain compare the average healthy population indicating that the increase dosage is adequate for maintaining the reference xanthophyll level in the brain. The subject will now continue with this dosage and be evaluated once every six months instead of every two months.

In one embodiment of any of the method described herein, the xanthophyll measured from the subject is Lutein (L). In another embodiment, the xanthophyll measured from the subject is a combination of L and zeaxanthin (Z). In yet another embodiment, the xanthophyll measured from the subject is Z. The discovery of a positive correlation between L and Z in the macula lutea or RBCs with those in the occipital cortex means that a measurement of the levels of L and Z in the macula lutea or RBCs can be used to accurately reflect the concentration of L and Z in other parts of the brains. The measured levels of L and Z are compared to reference L and Z levels respectively in order to make a diagnostic or dietary supplement evaluation.

In one embodiment of any of the method described herein, the reference level of xanthophyll, specifically L and Z, is that level of xanthophyll in a population of healthy humans with a mean age=65 years old. These healthy seniors have not been diagnosed with MCI by any methods known in the art and they do not have any symptoms known to be associated with MCI. A population comprises ten subjects or human subjects or more and can comprise both genders. In one embodiment, the reference level of xanthophyll is obtained from one gender type. The reference level of xanthophyll is measured by the same method as that used for measuring the xanthophyll level in the subject described herein.

In one embodiment, the reference level of xanthophyll (L plus Z) is about 0.35 MPOD unit when the macula is evaluated. In one embodiment, a MPOD unit above about 0.35 is the level of xanthophyll in the brain that is neuroprotective. A subject with an MPOD unit lower than about 0.0315 unit (lower by 10% of 0.35) is considered to be affected with MCI and should be further evaluated by a physician and/or advice to intake L and Z dietary supplement.

In one embodiment, the reference level of xanthophyll (L plus Z) is about 0.50 ng/gram when RBC is evaluated. In one embodiment, a level of xanthophyll above about 0.50 ng/gram is the level of xanthophyll in the brain that is neuroprotective. A subject with a level of xanthophyll lower than about 0.45 ng/gram (lower by 10% of 0.5) is considered to be affected with MCI and should be further evaluated by a physician and/or advice to intake L and Z dietary supplement.

L and Z absorb blue light and therefore appear yellow at low concentrations and orange-red at high concentrations. In one embodiment, the measuring of xanthophyll (L and/or Z) is performed using light absorption of xanthophylls in a densitometer, and the wavelength of the absorbed light is between 450-495 nm.

In one embodiment of any of the method described herein, the measuring of xanthophyll is performed using heterchromatic flicker photometry (HFP).

In one embodiment of any of the method described herein, the measuring of xanthophyll is performed by measuring the macular pigment optical density (MPOD) of the macula lutea. The MPOD measures a combination of both L and Z in the macula pigment.

In one embodiment of any of the method described herein, the xanthophyll level is measured from RBCs and the xanthophyll is extracted from the RBCs with an organic solvent. The organic solvent can be any that is known in the art and is not limited to those described herein.

In one embodiment of any of the method described herein, the organic solvent used for extracting the RBCs is selected from a group consisting of a mixture of chloroform, methanol and hexane; a mixture of hexane, ethanol and ether; a hexane-tetrahydrofurane mixture; or a mixture of ethanol and hexane. In another embodiment of any of the method described herein, the organic solvent used for extracting the RBCs is selected from a group consisting of methanol, ethanol, octanol, capryl, propyl alcohol, and butyl alcohol. Various combinations of methanol, ethanol, octanol, capryl, propyl alcohol, and butyl alcohol are also contemplated. One skilled in the art would be able to adjust and determine the combination for the fatty acid extraction from cell membranes.

In one embodiment of any of the method described herein, the organic solvent used for extracting the RBCs is selected from a group consisting essentially of a mixture of chloroform, methanol and hexane; a mixture of hexane, ethanol and ether; a hexane-tetrahydrofurane mixture; or a mixture of ethanol and hexane. In another embodiment of any of the method described herein, the organic solvent used for extracting the RBCs is selected from a group consisting essentially of methanol, ethanol, octanol, capryl, propyl alcohol, and butyl alcohol. Non organic solvent additives can be added to enhance or speed up the extraction process or aid in phase separation, e.g. enhance the separation of aqueous and organic phase by “flatten” out the interphase region.

In one embodiment of any of the method described herein, the level of the extracted xanthophylls is measured by high performance liquid chromatography (HPLC).

In one embodiment of any of the method described herein, the subject is 65 years old or older. In one embodiment of any of the method described herein, the subject is 40 years old or older. In another embodiment of any of the method described herein, the subject has a family history of neurodegenerative diseases such as Alzheimer's, dementia, etc.

In one embodiment of any of the method described herein, the subject is not currently affected with cancer, Alzheimer's disease, vascular disease that can lead to vascular dementia, Creutzfeldt-Jakob disease, abnormal thyroid stimulating hormone, vitamin B12 deficiency, syphilis, sleep apnea, or Parkinson disease. These are examples of some medical conditions that can present MCI-like symptoms. However, when the underlying medical causes are identified, addressed, corrected and brought under control, the associated MCI-like symptoms often consequently disappears.

There are a number of contribution factors and/or causes of MCI and also MCI-like symptoms. These include at least diet, wherein participants with the highest total fat intake were found to have a significantly elevated relative risk of dementia. An increased risk of dementia was also associated with a high dietary intake of saturated fat and cholesterol.

Another factor is inflammation, wherein people who had both metabolic syndrome and high inflammation levels were more likely to experience cognitive impairment than were patients who suffered from neither.

Yet another factor is free radical damage. Free radicals are highly unstable molecules that react with other molecules in a damaging process known as oxidation. Areas of the body with high energy output, such as the brain, are particularly vulnerable to damage from free radicals. The body normally defends itself against the harmful effects of free radicals with antioxidants, including superoxide dismutase and glutathione peroxidase, as well as vitamins C and E. Animal studies have suggested that diets high in antioxidants can delay age-related memory loss.

Low carotenoid levels could play a role in cognitive impairment and vascular disease.

In addition, atherosclerosis that occurs in the arteries serving the brain (cerebrovascular disease) can reduce blood flow to the aging brain and increase the risk of stroke. The decreased blood flow can cause nerve cells in the brain to be lost prematurely, and consequently, mental function may decline. Still an additional factor is stress. Studies have shown that older men with elevated levels of epinephrine (a stress hormone) are more likely to suffer from mild cognitive impairment than are their peers with normal levels. It has also been shown that everyday stresses combined with major stressful events may exert a cumulative effect over a lifetime that exacerbates cognitive decline

Dehydroepiandrosterone deficiency is yet another factor. Dehydroepiandrosterone (DHEA) levels naturally decline as people age. Numerous studies have connected lowered DHEA levels to memory loss and decreased cognitive function.

Thyroid hormone levels are another factor. Hypothyroidism (low levels of thyroid hormone) is associated with poor concentration, memory disturbances, and depression. Low levels of thyroid hormone have also been linked to impaired cognitive function; chronic kidney disease; and a variety of neurodegenerative diseases such as dementia, delirium, Alzheimer's disease, by far the most common cause of dementia.

Accordingly, at least a subject who is obese, under high stress, chronically lacks in sleep, has vascular disease, or has a family history of Alzheimer's disease or senile dementia is at risk of developing MCI as the subject ages.

In some embodiments, the subject is at risk of developing MCI.

In one embodiment of any of the method described herein, the subject at risk of developing MCI is obese, under high stress, chronically lacks in sleep, has vascular disease, diabetes or has a family history of Alzheimer's disease or senile dementia.

In other embodiments of any of the method described herein, the early diagnosis or dietary xanthophyll supplement evaluation of the present invention is followed up by any of the conventional neuropsychological screens known in the art such as MMSE, TMTA, TMTB, Digit Symbol Substitution (DDS), Finger Tapping Test (FTT), Clock Drawing Test (CDT) and Word Fluency Test (WFT). Other clinical tests include but not limited to measuring cerebrospinal fluid (CSF) concentrations of two of the disease's biochemical hallmarks—amyloid beta42 peptide and tau protein.

In other embodiments of any of the method described herein, the dietary xanthophyll supplement comprises L and/or Z supplements in the range of 1-12 mg per day.

### Lutein and Zeaxanthin

Lutein (L) and zeaxanthin (Z) belong to the xanthophyll class of carotenoids, also known as oxycarotenoids, which are natural fat-soluble yellowish pigments. L and Z are derived exclusively from dietary sources, such as dark green leafy vegetables and orange and yellow fruits and vegetables. Dietary sources of these dihydroxycarotenoids include corn, egg yolks, broccoli, green beans, green peas, brussel sprouts, cabbage, kale, collard greens, spinach, lettuce, kiwi and honeydew. The xanthophylls, which in addition to L and Z, include alpha- and beta-cryptoxanthin, contain hydroxyl groups, which makes them more polar than other carotenoids. Although lutein and zeaxanthin have identical chemical formulas and are isomers, they are not stereoisomers. They are both polyisoprenoids containing 40 carbon atoms and cyclic structures at each end of their conjugated chains.

Carotenoids are naturally-occurring pigments which are synthesized by plants, algae, bacteria, and certain animals, such as birds and shellfish, and they have antioxidant activities. therefore protect against the damage caused by free radicals. Free radicals are harmful molecules that are produced through normal body processes, such as oxygen metabolism. Environmental sources of free radicals include cigarette smoke, air pollutants, radiation, certain drugs and environmental toxins. These dietary carotenoids can also filter damaging high energy blue wavelength light from the visible-light spectrum by as much as 90%.

Carotenoids are a group of hydrocarbons (e.g., carotenes) and their oxygenated, alcoholic derivatives (e.g., xanthophylls), and include, for example, actinioerythrol, astaxanthin, bixin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotene, β-cryptoxanthin, lutein, lycopene, violerythrin, zeaxanthin, and esters of hydroxyl- or carboxyl-containing members thereof. As a result of a high intake of fruits and vegetables, 34 carotenoids and their metabolites are found in human serum and tissues at varying concentrations. Alpha-carotene, β-carotene, lycopene, lutein, β-cryptoxanthin, and zeaxanthin are the predominant carotenoids found in plasma. L and Z are the only carotenoids found in the macula of the human eye.

The human body can not make L, and zeaxanthin can only be made from L by the human bodies. Therefore, the lutein that is absolutely required for good vision must be obtained from the diet. Zeaxanthin must be obtained either from the diet or be synthesized in the body from dietary lutein. Therefore, by definition, lutein is in reality an essential vitamin.

L and Z are transported within the blood primarily on the surface of HDL (about 53%), but also on LDL (about 31%) and VLDL (about 16%). When these lipoproteins reach retinal tissue, they are transferred to that tissue by means of lipoprotein receptors found at the surface of RPE and Muller retinal cells. Although the precise mechanism has not yet been established, there is increasing evidence suggesting that HDL might be the most significant carrier for the retina. For example, within the plasma most (>60%) of apolipoprotein (Apo) E is associated with the HDL fraction. ApoE can be synthesized directly within the retina (Muller cells) and binds to receptors on ganglion cells. The subspecies of HDL containing ApoE (HDL-E) supplies lipids and lipid-soluble L and Z, to the retina.

The L and Z are concentrated in the macula of the human eye. While over 15 different dietary carotenoids are detectable in human serum, only L and Z and their metabolites are found to any substantial extent in the retina where they exist in approximately 500-fold higher concentrations than other body tissues (Bone R A and Landrum J T, 1992, Methods in Enzymology. San Diego: Academic, pages 360-366; Schmitz H H et al., 1993, Methods in Enzymology, 214:102-116). The macula or macula lutea (“yellow spot” in Latin) is an oval yellow area near the center of the retina of the human eye. At the centre of the macula is the fovea: with the largest concentration of cone cells (responsible for detailed vision and colour vision) in the eye and is responsible for central vision. Within the macula is the fovea—a small depression in the macula—which contains a high density of cones (photoreceptors with high acuity). Zeaxanthin concentration is highest in the center of the fovea, whereas lutein is relatively abundant in the perifoveal region.

Three carotenoids: L, Z and Meso-Zeaxanthin make up the macular pigment (MP) in the macula. Meso-Zeaxanthin is obtained by an enzyme conversion of lutein to meso zeaxanthin in the macula. It is not found in a typical diet. The MP has an important and naturally occurring protective function in the eye. The absorption spectra of L and Z enable these MPs to absorb blue light, which the can damage the retina (Snodderly D M., Am J. Clin. Nutr. 1995, 62:1448 S-1461S). Scattering and chromatic aberration of blue light can be minimized by these MPs. In addition, these carotenoids are also potent antioxidants, capable of decreasing lipid bilayer and DNA damage in membranes exposed to photooxidative and chemooxidative stressors (Sujak A, et al., Arch. Biochem. Biophys. 1999, 371:301-307), and that they are capable of absorbing highly energetic short-wave light (Krinsky N I., 2002, J. Nutr., 132:540 S-542S). Therefore, Z and L can protect the retina against oxidative damage in the macula where free radicals can be generated by lengthy light exposure, high oxygen tension, and high metabolic rate. Epidemiologic studies have shown that people with higher dietary or plasma lutein/zeaxanthin have reduced risk for advanced stages of retinal degeneration. The intensity of the yellow spot in the eye is proportional to its content of L plus Z, and this intensity is known as the macular density. Macular density progressively decreases as macular degeneration progresses due to loss of L plus Z.

A growing body of literature supports this finding in vivo (Moeller S M, et al., Arch. Ophthalmol. 2006, 124:1151-1162; SanGiovanni J P, et al., Arch. Ophthalmol. 2007, 125:1225-1232; Robman L, et al., Can. J. Ophthamol. 2007, 42:720-726; Parisi V, et al., Ophtalmology, 2007; Cangemi T E., BMC Ophthalmology 2007, 7:3; Falsini B, et al., Ophthalmology 2003, 110:51-62). Although the molecular basis of these neuroprotective effects of xanthophylls remains unknown, several mechanisms have been proposed such as (i) decreased oxidative stress, (ii) activation of anti-inflammatory pathways (Calder P C., Proc. Nutr. Soc. 2002, 61:345-358; Kritchevsky S B, Amer. J. Epidemiol., 2000, 152:1065-1071; Johnson E J., Nutr. Rev. 2005, 63:9-15; Price PT and Nelson C M., Curr. Opin. Lipidol. 2000, 11:3-7), (iii) modulation of functional properties of the synaptic membranes along with changes in their physicochemical and structural features (Gruszecki W I. in Carotenoids in Health and Disease. New York: Marcel Dekker, Inc, Krinsky N I, Mayne S T, Sies H, eds., 2004:151-163; Neuringer M, et al., Annu. Rev. Nutr. 1988, 8:517-41), thereby improving neural efficiency.

Although much recent work has focused on L and Z and their role in ocular health, L and Z are also the dominant carotenoids in various cortical regions, such as the frontal and occipital cortices, where they account for approximately 66-77% of total carotenoid concentration (Craft N E, et al., The Journal of Nutrition, Health & Aging, 2004, 8:156-162.). The function of carotenoids in the cortex is unknown. The most likely possibility is that L and Z in the cortex serve similar functions to L and Z in the neural retina. Consequently, cortical L and Z are likely also protective in nature and may also influence inter-neuronal communication and function via multiple mechanisms. Epidemiological evidence from the cognitive impairment literature suggests that this might be the case. For example, plasma L and Z, are depleted in both individuals with mild cognitive impairment (19, 20) and those with Alzheimer's disease (Rinaldi P., et al., Neurobiology of Aging 2003, 24:915-919).

Data in a 9-year study of over 500 elderly people have shown an inverse correlation of plasma carotenoid levels and cognitive performance in the elderly population (Akbaraly et al., 2007, J. Gerontology Series A: Biological Sciences and Medical Sciences 62:308-316). Healthy adults of mean age of 65 who score in the 10 and 25 percentile in Mini-Mental State Examination (MMSE) and score in the 70 and 90 percentile for the Trail Making Test Part A (TMTA) and the Trail Making Test Part B (TMTB) have lower plasma L and Z, well below the average for the elderly population of healthy 65 year olds of 0.27+0.15 μM and 0.032+0.021 μM respectively

### Cognition Function Monitoring

In some aspects, cognition function in an elderly individual can be evaluated and/or monitored with any methods known to a skilled artisan and described herein.

Improved memory and/or cognition can be measured following supplementation through a battery of genitive tests for memory and processing speed, or attention. The battery of cognitive tests included tests of memory and processing speed or attention and a measure of self-reported mood. All of these tests or versions of them have been used in cognitive aging research and also have demonstrated sensitivity to drugs or other health variables in treatment or epidemiological studies (See, for example, Ferris, S. H, et al. (1986). Assessing cognitive impairment and evaluating treatment effects: Psychometric performance tests. In L. Poon (Ed.), Handbook for Clinical Memory Assessment of Older Adults (pp. 139-148). Washington, D.C.: American Psychological Association; Letz, R. (1991). NES2 User's Manual (Version 4.4), Winchester, Mass.: Neurobehavioral Systems, Inc.; Johansson, B. & Zarit, S. H. (1997). Early cognitive markers of the incidence of dementia and mortality: A longitudinal population-based study of the oldest old. International Journal of Geriatric Psychiatry, 12, 53-59; Payton, M., Riggs, K. M., Spiro III, A., Weiss, S. T. & Hu, H. (1998). Relations of bone and blood lead to cognitive function: The VA Normative Aging Study. Neurotoxicology and Teratology, 20, 1-9). Alternate forms of Verbal Fluency and memory tests were administered at test sessions in order to decrease practice effects.

Verbal Fluency Test: Subjects name as many items from a category as possible during a one-minute period. (See, for example, Borkowski, J. G., Benton, A. L., & Spreen, O. (1967). Word fluency and brain damage. Neuropsychologic 5, 135-150).

Digit Span Forward and Backward: Subjects are asked to repeat numbers in increasing spans in forward sequences, then in backward sequences. Protocol adapted from Wechsler, D. A. (1981). Manual for the Wechsler Adult Intelligence Scale—Revised. New York: Psychological Corporation.

Shopping List Task: Ten associated words (common food items found in a supermarket) are read to the subject in up to five verbally presented serial trials. Verbal recall is tested immediately after each trial and after a delay (McCarthy, M., et al. (1981). Acquisition and retention of categorized material in normal aging and senile dementia. Experimental Aging Research, 7 127-135.)

Word List Memory Test: Ten unassociated words are presented (at a rate of one word every two seconds) on a computer monitor in three serial trials. Verbal recall is tested immediately after each trial and after a delay. (Computer version of test described by Morris, J. C, et al. (1989). The consortium to establish a registry for Alzheimer's disease (CERAD). Part I. Clinical and neuropsychological assessment of Alzheimer's disease. Neurology, 39, 1159-1165.)

MIR (Memory in Reality) Test: Subjects place 10 common household objects in 7 rooms of a model of an apartment. Verbal and visuospatial (location) recall is tested after a delay. (For protocol, see Johansson, B. (1988/89). The MIR—Memory-in-Reality Test. Psykologiforlaget AB, Stockholm.)

NES2 Pattern Comparison Test: Subjects choose the odd pattern from three similar patterns displayed on a computer monitor. The scores are the number of correct responses (maximum 15) and the mean response latency for correct decisions. (See Letz, R. (1991). NES2 User's Manual (Version 4.4), Winchester, Mass.: Neurobehavioral Systems, Inc.)

Stroop Test: Subjects name words (subtask 1—read words printed in black, and sub task 2—read color name words printed in the same color) and colors (subtask 3—name colors of rectangles, and subtask 4—name colors in which color name words are printed, when colors are different from the color name) in this assessment of response time and the ability to inhibit non-salient information. This version is presented via computer. Protocol adapted from Stroop, J. R. (1935). Studies of interference in serial verbal reactions. Journal of Experimental Psychology, 18, 643-662.

NES2 Mood Scales: Subjects rate their degree of tension, depression, anger, fatigue, and confusion over the previous seven days, using a computerized format. The Mood Scales are adapted from the Profile of Mood States (POMS; McNair, Lorr, & Dropleman, 1971). For description, see Letz, R. (1991). NES2 User's Manual (Version 4.4), Winchester, Mass.: Neurobehavioral Systems, Inc.

In addition, commonly used tests to monitor dementia such as the Wechsler Adult Intelligence Scale and the Cambridge Cognitive Test (CAMCOG) can be used. These tests have a number of different sections and test a variety of things, including the ability to learn new things and the ability to comprehend arithmetic and vocabulary.

In other embodiments, cognition function can be assessed by any of the conventional neuropsychological screens known in the art such as MMSE, TMTA, TMTB, Digit Symbol Substitution (DDS), Finger Tapping Test (FTT), Clock Drawing Test (CDT) and Word Fluency Test (WFT).

### Sample Collection and Preparation

Collections of samples comprising L and/or Z can be performed by methods well known to those skilled in the art. In some embodiments, the samples comprising L and/or Z include but are not limited to peripheral circulating whole blood, plasma, serum, red blood cells (RBC), platelets, and white blood cells (WBC).

For example, the patient's blood can be drawn by trained medical personnel directly into anti-coagulants such as citrate, EDTA PGE, and theophylline. The whole blood can be separated into the plasma portion, the cells, and platelets portion by refrigerated centrifugation at 3500×G for 2 minutes. After centrifugation, the supernatant is the plasma and the pellet is RBC. Since platelets have a tendency to adhere to glass, it is preferred that the collection tube be siliconized.

Another method of isolating RBCs is described in Best, C A et al., 2003, J. Lipid Research, 44:612-620.

Alternatively, serum can be collected from the whole blood. Collect the blood in a hard plastic or glass tube; blood will not clot in soft plastic. Draw 15 mL of whole blood for 6 mL of serum. The whole blood is allowed to stand at room temperature for 30 minutes to 2 hours until a clot has formed. Carefully separate clot from the sides of the container using a glass rod or wooden applicator stick and leave overnight at 4° C. After which, decant serum, centrifuge, and/or using a Pasteur pipette, remove serum into a clean tube. Clarify the serum by centrifugation at 2000-3000 rpm for 10 minutes. The serum can be stored at −20° or −80° C. before analysis. Detailed described of obtaining serum using collection tubes can be found in U.S. Pat. No. 3,837,376 and is incorporated by reference. Blood collection tubes can also be purchased from BD Diagnostic Systems, Greiner Bio-One, and Kendall Company.

In one embodiment, platelets are separated from whole blood and the levels of L and Z are determined there from. When whole blood is centrifuged as described herein to separate the blood cells from the plasma, a pellet is formed at the end of the centrifugation, with the plasma above it. Centrifugation separates out the blood components (RBC, WBC, and platelets) by their various densities. The RBCs are denser and will be the first to move to the bottom of the collection/centrifugation tube, followed by the smaller white blood cells, and finally the platelets. The plasma fraction is the least dense and is found on top of the pellet. The “buffy coat” which contains the majority of platelets will be sandwiched between the plasma and above the RBCs. Centrifugation of whole blood (with anti-coagulant, PGE and theophylline) can produce an isolated a platelet rich “buffy coat” that lies just above the buoy. The buffy coat contains the concentrated platelets and white blood cells.

In another embodiment, platelets can be separated from blood according to methods described in U.S. Pat. No. 4,656,035 using lectin to agglutinate the platelets in whole blood. Alternatively, the methods and apparatus described in U.S. Pat. No. 7,223,346 can be used involving a platelet collection device comprising a centrifugal spin-separator container with a cavity having a longitudinal inner surface in order to collect the “buffy coat” enriched with platelets after centrifugation. As another alternative, the methods and apparatus as described in WO/2001/066172 can be used. Each of these references is incorporated by reference herein in their entirety.

In another embodiment, platelets can be isolated by the two methods described in A. L. Copley and R. B. Houlihan, Blood, 1947, 2:170-181, which is incorporated by reference herein in its entirety. Both methods are based on the principle that the platelet layer can be obtained by repeated fractional centrifugation.

The whole blood can be first separated into platelet-rich plasma and cells (white and red blood cells). Platelet rich plasma (PRP) can be isolated from the blood centrifugation of citrated whole blood at 200 g for 20 minutes. The platelet rich plasma is then transferred to a fresh polyethylene tube. This PRP is then centrifuged at 800 g to pellet the platelets and the supernatant (platelet poor plasma [PPP]) can be saved for analysis by ELIZA at a later stage. Platelets can be then gently re-suspended in a buffer such as Tyrodes buffer containing 1 U/ml PGE2 and pelleted by centrifugation again. The wash can be repeated twice in this manner before removing the membrane fraction of platelets by centrifugation with Triton X, and lysing the pellet of platelet for platelet-derived PF4 analyses. Platelets can be lysed using 50 mM Tris HCL, 100-120 mM NaCl, 5 mM EDTA, 1% Igepal and Protease Inhibitor Tablet (complete TM mixture, Boehringer Manheim, Indianopolis, Ind.).

In some embodiments, apparatus and related methods are used to obtain the sample, for example, machines described in U.S. Pat. Nos. 4,120,448, 5,879,280 and 7,241,281, and they are incorporated by reference.

In some embodiments, the xanthophylls in the sample are extracted before measurement by methods known in the art and described herein. The xanthophylls can be extracted by any methods known it the art, for example, with organic solvents such as hexane-ether mixture described herein, methanol, ethanol, octanol, capryl, propyl alcohol, butyl alcohol or an ethanol-hexane mixture described in U.S. Pat. Nos. 5,847,238 and 6,169,217; Jose Luis Navarrete-Bolaños et al. 2005, Food Research International Volume 38:159-165; Elizabeth J. Johnson, et al. 2005, IOVS, February 2005, 46:693-702; and Kale A. and Cheryan M. 2007, J. Liquid Chromatography & related technologies, 30: 1093-1104.

### Determination of Lutein and/or Zeaxanthin

In one embodiment, the lutein and/or zeaxanthin are measured according to methods to one skilled in the art, for example, as described in Akbaraly et al., 2007, The Journals of Gerontology Series A: Biological Sciences and Medical Sciences 62:308-316; by Yeum et al. (Yeum, K. J., et A., Am J Clin Nutr, 1996. 64(4): p. 594-602) and in Nakagawa K. et al., 2008, Anal. Biochem. 381:129-34. Briefly, lutein and/or zeaxanthin are measured with a Biotek-Kontron high-performance liquid chromatography (HPLC) system (UVK Lab, Trappes, France), which consists of a 525 dual pump, a 465 autosampler, and a 540 diode array detector. Standards of lutein and/or zeaxanthin can be purchased from Fluka (Sigma-France, L′Isle d'Abeau), or Hoffman-La Roche (Hoffman-La Roche, Bale, Switzerland). The LC separation is run with an Alltech Adsorbosphere C18 coluMCI (150×4.5 mm ID, 3 μm particle size; Templemars, France), which was thermostated at 28° C. with a 402 coluMCI oven. The lutein and/or zeaxanthin are measured by HPLC after two extractions with a hexane-tetrahydrofurane mixture. For quantification, the method of Steghens and colleagues (J. Chromatogr. B. Biomed. Sci. Appl. 1997, 694:71-81). Internal controls were analyzed in every series of measurements (one control every 10 unknown samples). The limits of detection is calculated as 5-fold the maximum baseline noise in the region of the peaks.

In some embodiments, the macular pigment optical density (MPOD) can be measured by any method known in the art, such as described in B. R. Wooten et al. 1999, Invest. Ophthal. Visual Science. 1999, 40:2481-2489; R. L. van der Veen, et al., 2009, Ophthalmic Physiol Opt, 29:127-37; by the MPS 9000 series developed by Tinsley Precision Instruments Ltd, UK; U.S. Pat. No. 4,937,764 7467870; and the MACUSCOPE™ from MacuChek. As described herein, the intensity of the yellow spot (macula lutea) in the eye is proportional to its content of L plus Z, and this intensity is known as the macular density.

The method of assessing MPOD involves a three-channel Maxwellian view optical system and used a psycho physical method described by Hammond et al. (Hammond B. R., et al., Vision Res, 1996. 36: p. 2001-2012). A rotating sectored mirror combined two channels to produce the test stimulus, which alternated between a measuring and a reference field. The third channel provided a 10 degree background field. Ditric Optics interference filters were used to set the wavelength of the background (470 nm) and reference (550 nm) channels. A grating monochromator (Bausch and Long, Model, No. HD426) was used to determine the wavelength of the measuring (460 nm) stimulus. The stimulus subtended 0.8 degrees of visual angle and was centrally fixated for measurements of peak MP density Additional measurements of macular pigment were obtained by having the subject look at a fixation point at 1.5°, 3° and 5° temporal retinal eccentricities. The fixation point was produced by a small black dot on transparent glass in the path of the light that formed the background field.

The parafoveal reference was located at 7° temporal retinal eccentricity. The subject's head position was stabilized with an adjustable bitebar and headrest apparatus. Thus, a profile of MPOD in the temporal retina was obtained for each subject. Measurements were made in the right eye for all subjects. MPOD was measured in two baseline sessions on separate days and in two sessions on separate days at the end of 4 months of supplementation.

In U.S. Pat. No. 7,467,870 is a reflectometry instrument includes a light source, a spectrometer, and a first and second lens. The light source emits an illumination beam to the macula. The spectrometer measures a detection beam that is a portion of the illumination beam reflected from the eye and is indicative of the eye characteristics (e.g. macular pigment). The first and second lenses transmit the illumination beam to the macula and transmit the detection beam from the macula to the spectrometer. The instrument is used on an undilated pupil and minimizes unwanted reflections by at least one of the following: the first and second lenses include anti-reflection coatings; the illumination and detection beams pass through the first and second lenses at locations offset from their centers; and the illumination and the detection beams remain separated when passing through the first and second lenses. Zeaxanthin, lutein, and the total macular pigment levels are measured by the instrument.

Heterochromatic flicker photometry (HFP) is a way of measuring the spectral sensitivity of the human eye. Two lights of different colour are sinusoidally alternated at, typically, 10-20 Hz, and their relative intensities adjusted by the observer until the sensation of flicker is minimized. This technique has been used to define the human photopic luminosity, or V lambda, function on which photometry is based. Measurement of the macular pigment optical density (MPOD) by heterochromatic flicker photometry (HFP) is accomplished by viewing a small circular stimulus that alternates between a test wavelength that is absorbed by the MP (typically—blue, 460 nm) and a reference wavelength that is not absorbed (typically—green, 540 nm). Flicker observed by the subject is reduced to a null point by adjusting the intensity of the former while viewing the stimulus centrally, and then peripherally. A higher intensity, I, of the blue component of the stimulus is needed under central viewing conditions owing to attenuation by the MR The MPOD at the test wavelength is given by log(Icentral/Iperipheral) (Bone R A and Landrum J T, 2004, Arch Biochem Biophy. 430:137-42).

### Systems for Early Detection, Diagnosis and Surveillance of Dietary Supplement Requirement

Embodiments of the invention also provide for systems (and computer readable media for causing computer systems) to perform a method for diagnosing MCI in a subject, monitoring a subject's risk of developing MCI by monitoring the xanthophyll level of a subject regularly over time, and for surveillance of dietary supplement requirement and adjustments.

In one embodiment, provided herein is a system comprising: (a) a measuring module quantifying a xanthophyll level comprising a signal from an absorption of light having a wavelength between 450-495 nm capable of indicating a level of xanthophyll in a subject; (b) a storage module configured to store data output from the measuring module; (c) a comparison module adapted to compare the data stored on the storage module with a reference and/or control data, and to provide a retrieved content, and (d) an output module for displaying the retrieved content for the user, wherein the retrieved content the level of xanthophyll is lower than the reference and/or control data indicates that the subject is affected with MCI. The xanthophyll level is lower than the reference and/or control data by about at least 10%, at least 20%, at least 30%, at least 40%, at least 50%, at least 60%, at least 70%, at least 80%, at least 90%, 100%, 200%, 300% or 1000%, including all the percentages between 10-1000%. In one embodiment, the lower xanthophyll level indicates that the subject is likely to have MCI and is encourage to be further evaluated by a physician.

In one embodiment, provided herein is a system to facilitate the diagnosis of MCI and/or monitoring development of MCI in a subject, comprising: (a) a determination module configured to receive and output a xanthophyll level obtained from a subject, wherein the xanthophyll level is measured by an absorption of light with a wavelength between 450-495 nm; (b) a storage module configured to store output data from the determination module; (c) a comparison module adapted to compare the output data stored on the storage module with a reference and/or control data, and to provide a comparison content, and (d) an output module for displaying the comparison content for the user, wherein if the measured level of xanthophyll from the subject is lower than the reference and/or control data indicates that the subject affected with MCI or if there is a reduction of about at least 10% to a prior reading, then the subject likely has developed MCI and is advised to take xanthophyll dietary supplement. The xanthophyll level is lower than the reference and/or control data by about at least 10%, at least 20%, at least 30%, at least 40%, at least 50%, at least 60%, at least 70%, at least 80%, at least 90%, 100%, 200%, 300% or 1000%, including all the percentages between 10-1000%.

In one embodiment of the system described herein, the control data comprises previous data from the same subject.

In one embodiment, provided herein is a computer readable storage medium comprising: (a) a storing data module containing data from a subject that represents as a signal from an absorption of light with wavelength between 450-495 nm indicating a level of xanthophyll; (b) a comparison module that compares the data stored on the storing data module with a reference data and/or control data, and to provide a comparison content, and (c) an output module displaying the comparison content for the user, wherein if the measured level of xanthophyll from the subject is lower than a reference level of xanthophyll indicates that the subject affected with MCI or if there is a reduction of at least 10% to a prior reading, then the subject likely has developed MCI and is advised to take xanthophyll dietary supplement. The xanthophyll level is lower than the reference and/or control data by about at least 10%, at least 20%, at least 30%, at least 40%, at least 50%, at least 60%, at least 70%, at least 80%, at least 90%, 100%, 200%, 300% or 1000%, including all the percentages between 10-1000%.

In one embodiment of any of the system described herein, the reference or control data comprises data from a plurality of subjects who do not have symptoms associated with MCI. In one embodiment of any of the system described herein, the reference or control data comprises data from a population of healthy subjects who do not have symptoms associated with MCI. In one embodiment, the subjects' average age is 65. In another embodiment, the subject's age is over 65. In one embodiment, a population comprises a minimum of ten healthy subjects who do not have symptoms associated with MCI.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a measuring module #40, a storage module #30, a comparison module #80, and an output module #110. The functional modules can be executed on one, or multiple, computers, or by using one, or multiple, computer networks. The measuring module has computer executable instructions to provide e.g., xanthophyll level, absorbance reading, MPOD units etc in computer readable form.

The measuring module #40, can comprise any system for detecting a signal representing xanthophyll level in the macula lutea or RBCs. In some embodiments, such systems can include a HFP densitometer and/or C18 reverse phase HPLC coupled to a spectrophotometer as the eluant detector having the wavelength between 450-495 nm.

The information determined in the determination system can be read by the storage module #30. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, xanthophyll level, MPOD or OD_{450-495 nm} information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the reference/control data stored in the storage module to be read by the comparison module is e.g., xanthophyll level, MPOD or OD_{450-495 nm} data obtained from a population of subjects who do not have symptoms associated with MCI, xanthophyll level, MPOD or OD_{450-495 nm} data obtained from the same subject at a prior time point using the measuring module #40.

The “comparison module” #80 can use a variety of available software programs and formats for the comparison operative to compare expression data determined in the measuring module to reference samples and/or stored reference data. In one embodiment, the comparison module is configured to use pattern recognition techniques to compare information from one or more entries to one or more reference data patterns. The comparison module can be configured using existing commercially-available or freely-available software for comparing patterns, and may be optimized for particular data comparisons that are conducted. In some embodiments, the comparison module provides computer readable information related to xanthophyll level, MPOD units or OD_{450-495 nm} data, presence/absence of MCI in an individual, and/or sufficiency of xanthophyll dietary supplement in an individual.

The comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module #110.

In some embodiments, the content based on the comparison result, can be an expression value compared to a reference showing the presence/absence of MCI in an individual or an assessed risk of a subject to developing MCI, the efficacy of the xanthophyll dietary supplement in an individual.

In one embodiment of the invention, the content based on the comparison result is displayed on a computer monitor #120. In one embodiment of the invention, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user can construct requests for retrieving data from the comparison module. Thus, the user will typically point and click to user interface elements such as buttons, pull down menus, scroll bars and the like conventionally employed in graphical user interfaces.

The present invention therefore provides for systems (and computer readable media for causing computer systems) to perform methods for diagnosing MCI, assessing risk of developing MCI, assessing the need to supplement a subject's diet with xanthophyll supplements or assessing dietary supplement efficacy of in a subject.

Systems and computer readable media described herein are merely illustrative embodiments of the invention for assessing the level of xanthophyll in a subject and diagnosing MCI/dietary supplement, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

This invention is further illustrated by the following example which should not be construed as limiting.

The contents of all references cited throughout this application, examples, as well as the figures and tables are incorporated herein by reference in their entirety.

### EXAMPLES

### Example 1

### The Relationship Between Mini Mental Exam Scores (MMSE) and Macular Pigment Optical Density (MPOD)

### Methods and Materials

### Participants

The Health, Aging and Body Composition (Health ABC) Study is a large, NIA-sponsored 10 year prospective epidemiologic study of weight, weight related health conditions and physical function in older adults. The Age-Related Maculopathy Ancillary (ARMA) Study was conducted in year 6 and 7 of the Health ABC investigation and was designed to address association between macular pigment (MP), carotenoids, inflammation and ocular status in a sample of the Memphis cohort. Participants in the ARMA study (n=341) were aged 69-91 years of age. Approximately equal numbers of males and females were enrolled, and a significant proportion of the sample was African American (−15%). Participants were free of AMD, type I and II diabetes for at least 10 years, glaucoma and inflammatory ocular or systemic inflammatory disorders. The data from only those subjects for whom complete cognitive assessment and MP density were obtained were included in the current analysis (n=99, M=77 years of age, SD=3 years). The protocol was approved by the University of Tennessee Institutional Review Board. Informed consent was obtained from each participant, and the tenets of the Declaration of Helsinki were adhered to at all times.

### MP Density

MP Optical Density (MPOD) was assessed via an LED-based heterochromatic flicker photometer (HFP) device (Macular Metrics Corp.; Rehoboth, Mass.) (Iannaccone A, Mura M, Gallaher K, Study. HA. Macular pigment optical density in the elderly: Fndings in a large, biracial Midsouth population sample. Invest Ophthalmol Vis Sci 2007; 48:1458-1465; Wooten B R, Hammond B R, Land R I, Snodderly D M. A practical method for measuring macular pigment optical density. Invest. Ophthalmol. Vis. Sci. 1999; 40:2481-2489). HFP is the most widely validated MP measurement technique to date and has been validated in elderly subjects (Iannaccone A, Mura M, Gallaher K, Study. H A. Invest Ophthalmol Vis Sci 2007; 48:1458-1465; Gallaher K T, Mura M, Todd W A, Study. H A. Vision Res. 2007 April; 47(9):1253-9. Epub 2007 Mar. 21; Snodderly D M, Mares. J. A., Wooten B R, Oxton L, Gruber M, Ficek T. Invest. Ophthalmol. Vis. Sci. 2004; 45:531-538; Mares J A, LaRowe T, Snodderly D M, et al. American Journal of Clinical Nutrition 2006; 84:1107-1122.). MPOD was assessed at 0.5-deg of eccentricity (via a 1-deg target) using a 7-deg parafoveal reference. MPOD was assessed in year 6 or 7 of the Health ABC Study. Further details about the testing methodology, the simplified protocol utilized in this elderly population, and its repeatability within it have been published elsewhere (Iannaccone A, Mura M, Gallaher K, Study. H A. Macular pigment optical density in the elderly: Fndings in a large, biracial Midsouth population sample. Invest Ophthalmol Vis Sci 2007; 48:1458-1465, Gallaher K T, Mura M, Todd W A, Study. H A. Vision Res. 2007 April; 47(9):1253-9. Epub 2007 Mar. 21).

### Cognitive Assessment

The Modified Mini-Mental State Examination was administered in study years 3, 5, and 7, with more extensive testing performed in years 5 and 7 (Teng. E. L., Chui H C. The Modified Mini Mental State (3MS) Examination. J. Clin. Psychiatry 1987; 48:314-318). Consequently, the average values from these latter two testing years were used in this present study. This test is a brief, general cognitive battery with components for orientation, concentration, language, praxis, and immediate and delayed memory. Possible scores range from 0 to 100, with higher scores indicating better cognitive function.

### Results

The aim of this study was to evaluate the relationship between macular pigment density and cognitive function in healthy older adults. Macular pigment is lutein and zeaxanthin embedded in neural (retinal) tissue. An intervention trial in healthy older adults found lutein supplementation to improve cognitive function (Johnson E J, McDonald K, Caldarella S M, Chung H-Y, Snodderly D M. Nutritional Neuroscience 2008, 11:75-83).

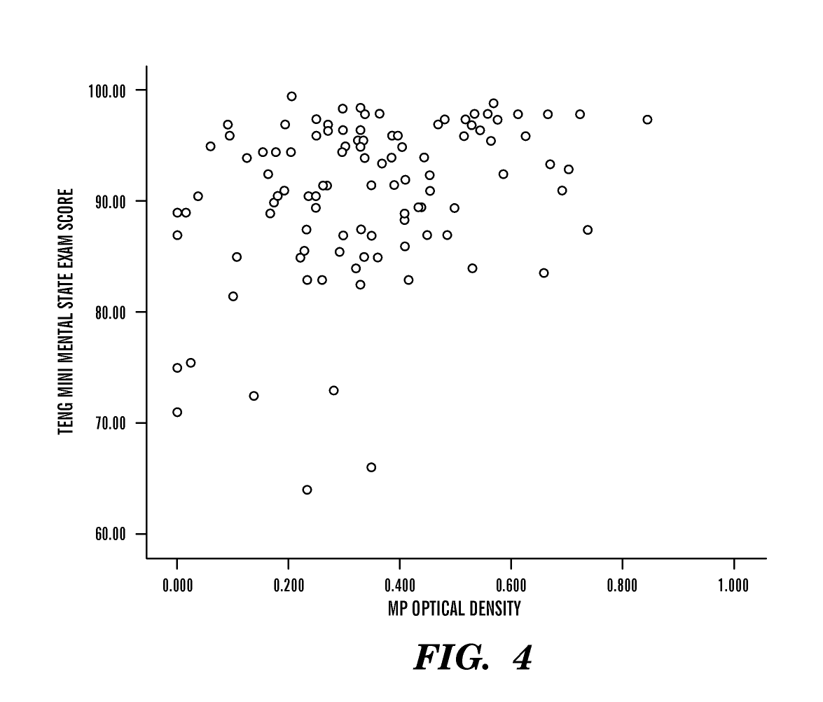

Results: Macular pigment optical density was significantly correlated with the MMSE scores (p=0.029). No low MMSE was observed for MPOD >0.37, with the eight lowest scores being below this level (FIG. 4).

### Example 2

### Relationship of Xanthophylls (Lutein & Zeaxanthin) in Plasma, Red Blood Cells, and Macula in Healthy Adults

### Introduction

There is continued interest in the study of dietary xanthophylls (lutein and zeaxanthin) because of the data that show that diets rich in these nutrients may reduce the risk of age-related macular degeneration (AMD) (Snodderly D M. Am J Clin Nutr 1995; 62:14485-14615). Intervention and cross-sectional studies suggest that xanthophylls may be important in cognitive health as well (Johnson E J, et al., Nutritional Neuroscience 2008; 11:75-83; Renzi L M, Iannaccone A, Gallaher K T, et al. The relation between serum xanthophylls, fatty acids, macular pigment and cognitive function in the Health ABC Study in preparation 2007.). Currently, dietary and plasma concentrations of xanthophylls are commonly used to assess status. The use of RBC membranes as a measure xanthophyll assessment has not been explored. Red blood cells (RBC) concentrations are the preferred method for fatty acid assessment (as opposed to plasma) because it is considered a long-term measure of fatty acid status. Similar to fatty acids, lutein and zeaxanthin are fat soluble and integrated in cell membranes (SanGiovanni J P, Chew E Y. Prog Retina Eye Res 2005; 24:87-138; Chew E Y, SanGiovanni J P. Lutein. In: Coates P, Blackman, M. R., Cragg, G., Levine, M., Moss J., White, J., ed. Encyclopedia of Dietary Supplements. London: Informa Healthcare, 2004). Thus, RBC concentrations of lutein and zeaxanthin may be a better measure of long-term xanthophyll intakes than plasma concentrations as evident by evaluations of correlations with macular pigment density presented herein. Macular pigment is lutein and zeaxanthin embedded in retinal tissue, which is considered a long-term measure of xanthophyll status (Whitehead A J, Mares J A, Danis R P. Archives of Ophthalmology 2006; 124:1038-45).

### Research Methods

Experimental design. This is a cross-sectional study in 15 healthy subjects recruited from the general population. Subjects were selected based on their intake of xanthophylls to get a large range of dietary lutein and zeaxanthin intakes. Blood and macular pigment density measures were collected from each subject. Laboratory analyses were conducted in the Carotenoids & Health Laboratory at the Jean Mayer USDA HNRCA at Tufts University.

Subjects. Fifteen healthy men (n=5) and women (n=10), 23-66 years were recruited for this study. There was no ethnicity exclusion. Subject eligibility and exclusions are the following:

Eligibility Criteria:
1. Healthy non smoking (within past 6 months) men and women (non pregnant, non-lactating)
2. Must be able to give written informed consent
3. No blood donation within the past 56 days.
4. Absence of fat malabsorption
5. No drug intake that interferes with fat absorption or metabolism of blood clotting

Exclusion Criteria:
1. Patients with a history of active small bowel disease or resection, atrophic gastritis, alcohol consumption 2 drinks/day or >14 drinks/week, pancreatic disease, digestive disease, e.g. Sprue, Crohn's, Celiac, diabetes (type I and II) eye disease (macular degeneration, cataracts, glaucoma) and bleeding disorders
2. Supplementation with lutein (>2 mg/d)
3. Body mass index (BMI)<20, ≧35 kg/m2
4. Hyperlipidemia

Inclusion/Exclusion criteria were determined by medical history and questionnaire. The questionnaire included questions on age, smoking status, chronic disease, medication use, supplement use, alcohol intake, eye disease, any high cholesterol (yes or no), problems with bleeding last date of blood donation.

Extraction of Carotenoids from Red Blood Cells (RBC)

Chemicals: sodium hydroxide (NaOH), SIGMA® Catalog No. S-5881; de-ionized water; hexane, HPLC grade; ethyl ether, Fisher Scientific (Catalog No. E197-1); ethanol, HPLC grade; hexane:ether (2:1) (v/v); 0.9% Sodium Chloride (saline)

RBC Preparation & Storage: Collect blood sample into tube containing anti-coagulant. For example, in with an 8% liquid EDTA solution. Commercially available blood collection tubes are commonly color coded to indicate the additives in the tube. For example, the lavender top blood collection tubes have calcium EDTA as an anti-coagulant. Spin at 3000 rpm (˜1000 g) for 10 min within 1 hr of blood collection. At least three distinct layers can be observed, a red pellet of RBCs at the bottom, a thin middle cream to whitish colored buffy coat on top of the red pellet and the clear yellow liquid component of blood as the top layer. Remove the buffy coat layer that is between the plasma and RBC layer, and store the plasma separately. Aliquot RBCs into freezer vials at ˜1 mL each and store at −70° C. till needed.

Extraction of lipid: Thaw RBC and vortex before pipetting. Add 500 mg RBCs (record mass) into a 16×100 mm screw-cap tube, add 1 mL saline and homogenize (model IKA®, 30 sec, speed 4). Rinse probe of the homogenizer with 1.5 mL ethanol and add to homogenate. Add 4 mL hexane:ether (2:1) to homogenate. Add 50 μL of internal standard (echinenone, OD455 ˜0.1: retinyl acetate, OD340 ˜0.1 in ethanol) and vortex. Centrifuge at 1000 g for 5 min and remove the upper organic layer and add to a 12×75 mm disposable culture tube. Dry the organic layer under N₂ in water bath (40° C.). To remaining upper layer, repeat hexane:ether extraction twice times. Combine extracts and dry under N₂. Resuspend the dried extract in 75 μL of ethanol, vortex and sonicate 30 seconds. Inject 25 μL onto HPLC for carotenoid analysis. On skilled in the art will be able to adjust the volume to resuspend the dried extract and also the amount to inject in the HPLC in order to obtain readings that are within the linear measurement range of the instrument.

### Collection Procedures and Analytical Techniques.

Blood Draw A single 20 mL fasting blood draw will be performed during the 1-day visit to the HNRCA. Plasma was separated from blood. Samples (plasma, RBC) were stored in the Carotenoids & Health Laboratory at the HNRCA at −70° C. until the time of analysis.

HPLC analysis for carotenoids Extraction of xanthophylls from plasma was performed as described elsewhere (Johnson E J, Hammond R B, Yeum K-J, et al. Am J Clin Nutr 2000; 71:1555-1562). Extraction of xanthophylls from RBC were performed using a method developed in our laboratory. Xanthophylls were measured using a reverse-phase, gradient HPLC system (Johnson E J, Hammond B R, Yeum K J, et al. Am J Clin Nutr 2000; 71:1555-62).

### Macular Pigment Optical Density (MPOD)

MPOD was measured using a psychophysical measurement as previously described by our laboratory (Hammond B R, Jr., Johnson E J, Russell R M, et al. Invest Ophthalmol Vis Sci 1997; 38:1795-801). This is a non invasive measure in which the subject is asked to perform a simple task of detecting a small flickering blue light. The procedure to this measure can be found at www.macularmetrics.com. The light levels to which the subject is exposed to during the eye test are well within accepted standards of safety and pupil dilation is not required. The measure takes 30-45 minutes. MPOD was assessed at 0.5-deg of eccentricity (via a 1-deg target) using a 7-deg parafoveal reference.

### Statistical Analysis:

Pearson correlation coefficients were used to measure the relationship between plasma and RBC and macular concentrations of xanthophylls (lutein+zeaxanthin).

### Results

Subject characteristics are given in Table 1. Both plasma and RBC concentrations of xanthophylls were significantly related to MPOD, however the strength of the relationship was stronger for RBC (FIG. 5). RBC measures of xanthophyll have a greater correlation with MPOD than measures in plasma. This could be due to plasma reflecting short-term intakes and RBC reflecting long-term intakes. The steeper slope indicates that RBC xanthophyll is more sensitive to fluctuations in MPOD, and therefore would be a better indicator of the levels of xanthophylls in the macula and in the brain.

TABLE 1
Subject Characteristics.

| | | BMI^{a} | Plasma L + Z^{b}, | RBC L + Z | MPOD^{c} 1.0 |
|---|---|---|---|---|---|
| | Age, yrs | kg/m² | μg/dL | ng/gram | degree |
| mean | 39 | 24.5 | 18.0 | 48.7 | 0.36 |
| SD | 16 | 3.3 | 9.2 | 27.7 | 0.13 |

^{a}BMI: body mass index;
^{b}L + Z: Lutein + Zeaxanthin;
^{c}MPOD: macular pigment optical density

### Example 3

The purpose of this study was to investigate the relationships between serum concentrations of carotenoids with measures of cognitive function in older adults.

Subjects: Participants were 326 centenarians or near-centenarians and octogenarians from the Georgia Centenarian Study (Poon L W, Jazwinski M, Green R, et al. Methodological consideration in studying centenarians. In: Poon L W, Perls, T. T., ed. Annual Review of Gerontology and Geriatrics. New York: Springer Publishing Company, 2007:231-264). All participants or their legal proxy provided informed written consent prior to participating. This study was approved by the University of Georgia Institutional Review Board. Most participants were assessed in their homes over four to five two-hour sessions. Subjects underwent cognitive examinations every 6 months until death. Physical and mental health, cognitive, functional, genetic, nutrition, resources, and personality data were collected as part of the larger study.

Measures: Cognition. Subjects were evaluated for various measures of cognitive function. The Mini-Mental State Examination (Folstein M, Folstein S E, McHugh P R, Fanjiang G. Mini-Mental State Examination user's guide. Odessa, Fla.: Psychological Assessment Resources., 2001) (MMSE) is a gross measure of cognitive status made up of 30 items that generate a score ranging from 0 to 30. The MMSE is widely used to assess orientation to time and place, immediate and short-delayed recall of words, attention, calculation, language, and visual construction. The Global deterioration rating scale (Reisberg B, Ferris S H, de Leon M J, Crook. T. The global deterioration scale for assessment of primary degenerative dementia. American Journal of Psychiatry 1982; 139:1136-1139) provides an assessment of the stage of cognitive function for those with a primary degenerative dementia, such as Alzheimer's disease. The Fuld Object Memory Evaluation (Fuld P A. Fuld Object Memory Evaluation. Chicago, Ill.: Stoetling, 1977) assesses memory to include delayed recall, delayed recognition and retention. Controlled oral word association test (Sumerall S W, Timmons P L, James A L, Ewing M J, Oehlert M E. Expanded norms for the Controlled Oral Word Association Test. Journal of Clinical Psychology 1997; 53:517-21) is a measure of verbal fluency. Weschsler Intelligence Scale (Wechsler D. Wechsler Adult Intellignece Scale-r. Revised manual. New York: Psychological Corp, 1981) measures adult intelligence. Behavioral dyscontrol scale (Grigsby J, Kaye K, Robbins L J. Reliabilities, norms and factor structure of the Behavioral Dyscontrol Scale. Perceptual & Motor Skills 1992; 74:883-92) is a brief measure of executive functioning. Geriatric depression scale is a 30-item self-report assessment used to identify depression in the elderly (Sheikh J I, Yesavage J A. Geriatric Depresion Scale:Recent evidence and development of a shorter version. Clinical Gerontology: A Guide to Assessment and Intervention. New York: The Haworth Press, 1986:165-173)

Serum Carotenoids. Serum carotenoid concentrations were analyzed via high-performance liquid chromatography using procedures described in our laboratory (Johnson E J, Hammond R B, Yeum K-J, et al. Relation among serum and tissue concentrations of lutein and zeaxanthin and macular pigment density. Am J Clin Nutr 2000; 71:1555-1562).

Data Analysis: All data were analyzed using SPSS 16.0 (SPSS Inc., Chicago). Data were adjusted for age, sex, BMI, smoking, alcohol, hypertension and diabetes. Pearson correlations were used to test the hypothesis that carotenoid status is related to cognitive function in older adults.

Results: Significant correlations were observed for most carotenoids with most measures of cognitive function (Table 2). Relationships were in the direction of better cognitive performance. Lutein was the only carotenoid to have serum concentrations that were significantly related to all measures of cognitive performance. Serum lutein concentrations also had the strongest correlations with cognitive performance.

Conclusions: Increased carotenoid status may be beneficial to cognitive health in the elderly. Among the carotenoids, lutein may be of particular benefit.

TABLE 2
Partial correlation coefficients between cognition indices and serum carotenoids adjusted
w/age, sex, BMI, smoking, alcohol, hypertension and diabetes (n = 326)

| | | | β-Crypto- | | |
|---|---|---|---|---|---|
| | Lutein | Zeaxanthin | xanthin | β-Carotene | Lycopene |
| Mini-mental state | 0.200* | 0.160** | 0.128*** | 0.219* | 0.180** |
| examination | | | | | |
| (global measure of | | | | | |
| cognition) | | | | | |
| Global deterioration | −0.362* | −0.228* | −0.173** | −0.274* | −0.300* |
| rating scale | | | | | |
| (measure of primary | | | | | |
| degenerative dementia) | | | | | |
| Delayed Recall | 0.287* | 0.165*** | 0.050 | 0.187** | 0.315* |
| (Fuld Object Memory | | | | | |
| Evaluation) | | | | | |
| Delayed Recognition | −0.134*** | −0.072 | 0.021 | 0.056 | −0.206** |
| (Fuld Object Memory | | | | | |
| Evaluation) | | | | | |
| Retention | 0.210** | 0.110 | 0.140*** | 0.259* | 0.055 |
| (Fuld Object Memory | | | | | |
| Evaluation) | | | | | |
| Controlled oral word | 0.253* | 0.142*** | 0.136*** | 0.168* | 0.209** |
| association test | | | | | |
| Wechsler Adult | 0.249* | 0.158*** | 0.201** | 0.125 | 0.205** |
| Intelligence Scale | | | | | |
| (wais) | | | | | |
| (measure of intelligenc | | | | | |
| quotient) | | | | | |
| Behavioral dyscontrol | 0.218* | 0.146*** | 0.147*** | 0.240* | 0.212** |
| scale | | | | | |
| (measure of executive | | | | | |
| function) | | | | | |
| Geriatric depression | −0.143*** | −0.076 | −0.132*** | −0.040 | −0.125 |
| scale-short form | | | | | |

*p < 0.001;
**p < 0.01;
***p < 0.05

### Example 4

### Serum Carotenoids as a Biomarker for Carotenoid Concentrations in the Brain

Carotenoids, specifically lutein and zeaxanthin, cross the blood brain barrier to form macular pigment in the eye. Macular pigment has been related to serum lutein and zeaxanthin. Lutein supplementation was shown to improve cognitive function in older adults possibly due to increase in brain lutein. The objective was to evaluate if serum carotenoids are related to brain carotenoids in older adults. Brain tissues (cerebellum, temporal, frontal and occipital cortices) from decedents who had participated in the Georgia Centenarian Study were used. Subjects (n=29) had agreed to donate their brain after death. Serum and brains were extracted and analyzed using RP-HPLC. Age-adjusted partial correlation was done using SYSTAT. P values <0.05 were considered significant. Serum lutein, lycopene and β-carotene were positively associated (P<0.05) with brain lutein, lycopene and β-carotene, respectively in all regions. Serum zeaxanthin was significantly associated with brain zeaxanthin only in the temporal and frontal cortices. Zeaxanthin in the cerebellum tended to correlate with serum zeaxanthin (P=0.071), while in the occipital cortex no association was observed. Cryptoxanthin in the temporal cortex tended to correlate with serum cryptoxanthin (P=0.066), but no association was observed in the other regions. Serum carotenoid concentrations can be used as a biomarker to predict brain carotenoid concentrations.

### Example 5

### Brain Levels of Lutein (L) and Zeaxanthin (Z) are Related to Cognitive Function in Older Adults

Background: Among the carotenoids, L and Z are preferentially taken up into human brain. Intervention with L has improved cognition in older adults.

Purpose: to evaluate the relationship between cognition and L and Z levels in brain tissue of decedents >80 yrs at death.

Methods: Subjects were from the Georgia Centenarian Study. Subjects (n=29) who agreed to donate their brains after death. Tissue included: cerebellum, frontal, occipital, and temporal cortices. Brain were analyzed with standard lipid extractions and reverse phase HPLC. Pearson correlations were performed using SPSS and data was corrected for age. Cognition measures included: global cognition, primary degenerative dementia, delayed recall, delayed recognition, retention, intelligence quotient, and executive function. P values <0.050 were considered significant.

Results: L levels in occipital and temporal cortices were significantly relation to retention. This was also true when L levels were combined with Z. Z alone in occipital cortex was significantly related to retention. L levels with and with out Z in occipital cortex tended to be related to global cognition (p<0.066) and with retention in the cerebellum and frontal and temporal cortices (p<0.089). L alone tended to be related to intelligence quotient in temporal cortex (p<0.10). Z alone was significantly related to retention in occipital lobe. There were no positive trends or significant relationships with primary degenerative dementia or executive functioning.

Conclusions: L and Z levels in the brain may be related to cognitive function in the elderly.

### Example 6

### The Relationship Between Xanthophyll Concentrations in Primate Retinal and Brain Tissue

The purpose of the research was to evaluate the relationship between xanthophyll (lutein and zeaxanthin) concentrations in primate retinal and brain tissue.

Methods: All procedures were approved by the Institutional Animal Care and Use Committee of the Oregon National Primate Research Center and conformed to NIH guidelines and the ARVO Statement for the Use of Animals in Ophthalmic and Vision Research.

Study 1. Lutein and Zeaxanthin Supplementation Study. Eighteen rhesus monkeys (Macaca mulatta) were reared on one of two semipurified diets, both of which contained adequate levels of all known nutrients, but no detectable xanthophylls as analyzed by our laboratory. The animals also received limited amounts of very low xanthophyll foods such as wheat or rice cereals, white rice, sweetened drinks and gelatin, pineapple and banana.

Beginning at 7-16 years of age, 6 of these monkeys were supplemented with pure lutein and 6 with pure zeaxanthin, at 3.9 μmol/kg/day (2.2 mg/kg/day). The lutein and zeaxanthin supplements were purified or synthesized by DSM Nutritional Products, Ltd. The supplements were inserted into marshmallows, sweetened gelatin or small pieces of fruit. Beadlets and individual supplement doses were stored at 4° C. in the dark. Supplements were provided daily for 4-12 months and, due to limited supply of the pure xanthophylls, 4×/week thereafter until the conclusion of the study. The duration of daily supplementation varied within each group but was matched between the two groups. However, because lutein had to be specially purified, its supply was more limited, resulting in a shorter duration of supplementation at 4×/wk for the lutein-fed group (3+1 mo compared to 7+2 mo for the zeaxanthin-fed group). The lutein-fed and zeaxanthin-fed groups were balanced to the extent possible based on sex, and body weight. The remaining 6 animals continued on their semipurified diets but received no xanthophyll supplements (xanthophyll-free). Two lutein-fed and 2 zeaxanthin-fed animals were sacrificed after 6-8 months of supplementation, 2 from each group at 13-14 months and two from each group each at 15-24 months.

Biopsy punches were used to dissect the central retina, containing the macula, into a 4 mm diameter circle centered on the fovea. Retinal tissue was analyzed for carotenoids as described previously (1). Cerebellum and occipital cortex samples were collected and analyzed for carotenoids as described by Park et al. (2). Due to technical difficulties, tissue was available for only 5 of the zeaxanthin-fed monkeys

Study 2. Standard Stock-Fed Monkeys: Normal control monkeys fed a standard stock diet (Purina 5047 Monkey Chow®) providing a daily carotenoid intake of 0.26 μmol/kg/day L, 0.24 μmol/kg/d Z and 0.035 μmol/kg/day β-carotene (means of four analyses). These animals also received supplemental fruits and vegetables (typically ¼-½ apple or ½ carrot, approximately 3×/week), which contributed an estimated maximum additional daily average of ˜3 nmol/kg of lutein plus zeaxanthin, or <1% of the intake from the stock diet. They were housed under the same conditions as the experimental diet groups.

Whole retinas were analyzed for carotenoids as described previously (Johnson E J, Neuringer M, Russell R M, Schalch W, Snodderly D M. Nutritional manipulation of primate retinas, III: Effects of lutein or zeaxanthin supplementation on adipose tissue and retina of xanthophyll-free monkeys. Investigative Ophthalmology & Visual Science 2005; 46:692-702.). Occipital cortex samples were collected and analyzed for carotenoids as described by Park et al. (Park J H, Hwang H J, Kim M K, Lee-Kim Y C. Effects of dietary fatty acids and vitamin E supplementation on antioxidant vitamin status of the second generation rat brain sections. Korean J. Nutrition 2001; 34:754-761.).

Relationships between retina and brain tissue were evaluated using Pearson's correlation coefficients. For those relationships with sample size >6, p values were calculated.

Results: No xanthophylls were detected in the tissues of monkeys fed a xanthophyll-free diet. In the lutein-supplemented monkeys lutein was the only carotenoid measured in brain tissue. In this group, there was a trend for lutein to be related in the cerebellum and retina (r=0.0654, p<0.079) and there was a significant relationship between lutein concentrations in the occipital cortex and retina (r=0.796, p<0.029) (FIGS. 1C and 1D). In the zeaxanthin-supplemented monkeys zeaxanthin was the only carotenoid measured in brain tissue. In this group, the correlation between retina concentrations of zeaxanthin and that in the cerebellum and occipital cortex was 0.789 and 0.623, respectively (FIGS. 1E and 1F).

Similar relationships were observed for the standard stock-diet monkeys (FIGS. 1A and 1B), with correlation coefficients of 0.975 and 0.978 for lutein and zeaxanthin, respectively.

Accordingly, we showed that retinal xanthophyll concentrations are related to cerebellum and occipital cortical xanthophyll concentrations. Thus, we discovered that measure of macular pigment in vivo can be used as a biomarker of xanthophylls incorporated in brain tissue.

What is claimed is:

1. A method of diagnosing of mild cognitive impairment (MCI) in a subject comprising measuring a xanthophyll level in macula lutea or in a red blood cell (RBC) sample from the subject and comparing the xanthophyll level with a reference xanthophyll level, wherein if the measured xanthophyll level from the subject is lower than the reference xanthophyll level the subject is affected with MCI.

2. The method of claim 1, wherein the xanthophyll is lutein.

3. The method of claim 1, wherein the xanthophyll is a combination of lutein and zeaxanthin.

4. The method of claim 1, wherein the xanthophyll is zeaxanthin.

5. The method of claim 1, wherein the measuring is performed using light absorption of xanthophylls in a densitometer, and the wavelength of the absorbed light is between 450-495 nm.

6. The method of claim 1, wherein the measuring is performed using heterchromatic flicker photometry (HFP).

7. The method of claim 1, wherein the xanthophyll level is measured from RBCs and the xanthophyll is extracted from the RBCs with an organic solvent.

8. The method of claim 7, wherein the organic solvent is selected from a group consisting of chloroform, methanol and hexane.

9. The method of claim 7, wherein the level of the extracted xanthophylls is measured by high performance liquid chromatography (HPLC).

10. The method of a claim 1, wherein the subject is 65 years old or older.

11. The method of claim 1, wherein the subject is not currently affected with cancer, Alzheimer's disease, dementia, Creutzfeldt-Jakob Disease, abnormal thyroid stimulating hormone, vitamin B 12 deficiency, syphilis, sleep apnea, or Parkinson disease.

12. The method of claim 1, wherein the reference xanthophyll level is an average level obtained from a population of healthy subjects without symptoms associated with MCI and is measured by the same method as that used for measuring the xanthophyll level in the subject.

13. A method of determining a need for a dietary supplementation of xanthophylls in a subject, the method comprising:
(a). measuring a xanthophyll level in a macula lutea or in a RBC sample from a subject at a first time point;
(b). measuring a xanthophyll level in the subject at a second time point using the same method as in step (a), wherein the xanthophyll measured in step (a) and (b) are the same;
(c). comparing the xanthophyll level obtained at the first time point with that obtained at the second time point, wherein if there is a decrease in the level of xanthophyll at the second time point compared to the first time point, the subject is in need of dietary supplementation of xanthophylls.

14. The method of claim 13, wherein the first and second time points are separated by 3-6 months.

15. The method of claim 13 further comprising measuring a level of xanthophyll at a third time point and comparing the level of xanthophyll obtained at the third time point with that obtained at the first and/or second time point, wherein the second and third time points are separated by 1-6 months, wherein if there is a decrease in the level of xanthophyll at the third time point compared to the first and/or second time point, the subject is in need of dietary supplementation of xanthophylls.

16. The method claim 13, wherein the xanthophyll is lutein.

17. The method of claim 13, wherein the xanthophyll is a combination of lutein and zeaxanthin.

18. The method of claims 13, wherein the xanthophyll is zeaxanthin.

19. The method of claim 13, wherein the xanthophyll level is measured using heterchromatic flicker photometry (HFP) using wavelength between 450-495 nm.

20. The method of claim 13, wherein the xanthophyll is measured from the RBCs and is extracted with an organic solvent.

21. The method of claim 20, wherein the organic solvent is selected from a group consisting of chloroform, methanol and hexane.

22. The method of claim 21, wherein the extracted xanthophyll is measured by high performance liquid chromatography (HPLC).

23. A method of monitoring the amount of dietary supplementation with xanthophylls in a subject taking xanthophyll supplements, the method comprising
(a). measuring a xanthophyll level in a macula lutea or in a RBC sample from a subject at a first time point;
(b). measuring a xanthophyll level in the subject at a second time point using the same method as in step (a), wherein the xanthophyll measured in step (a) and (b) are the same;
(c). comparing the xanthophyll level obtained at the first time point with that obtained at the second time point, wherein if there is a decrease in the level of xanthophyll at the second time point compared to the first time point, the subject is in need of increase in the dietary supplementation of xanthophylls.

24. The method of claim 23, wherein the subject is 65 years old or older.

25. The method of claim 23, wherein the subject has not been diagnosed with cancer, Alzheimer's disease, dementia, Creutzfeldt-Jakob Disease, abnormal thyroid stimulating hormone, vitamin B12 deficiency, syphilis, sleep apnea, or Parkinson disease.

26. The method of claim 23, wherein the subject is at risk of developing MCI.

27. The method of any claim 23, wherein the subject has a vascular disease.

28. A system comprising:
(a). a measuring module quantifying a xanthophyll level comprising a signal from an absorption of light having a wavelength between 450-495 nm capable of indicating a level of xanthophyll in a subject;
(b). a storage module configured to store data output from the measuring module;
(c). a comparison module adapted to compare the data stored on the storage module with a reference and/or control data, and to provide a retrieved content, and
(d). an output module for displaying the retrieved content for the user, wherein the retrieved content the level of xanthophyll lower than the reference and/or control data indicates that the subject is affected with MCI.

29. The system of claim 28, wherein the control data comprises data from a population of a population of healthy subjects who do not have symptoms associated with MCI.

30. A computer implemented system to facilitate the diagnosis of MCI and/or monitoring development of MCI in a subject, comprising:
(a). a determination module configured to receive and output a xanthophyll level obtained from a subject, wherein the xanthophyll level is measured by an absorption of light with a wavelength between 450-495 nm;
(b). a storage module configured to store output data from the determination module;
(c). a comparison module adapted to compare the output data stored on the storage module with a reference and/or control data, and to provide a comparison content, and
(d). an output module for displaying the comparison content for the user, wherein if the measured level of xanthophyll from the subject is lower than the reference and/or control data indicates that the subject affected with MCI or if there is a reduction of at least 10% to a prior reading, then the subject likely has developed MCI and is advised to take xanthophyll dietary supplement.

31. The computer implemented system of claim 30, wherein the control data comprises previous data from the same subject.

32. A computer readable storage medium comprising:
(a). a storing data module containing data from a subject that represents as a signal from the absorption of light with wavelength between 450-495 nm indicating a level of xanthophyll;
(b). a comparison module that compares the data stored on the storing data module with a reference data and/or control data, and to provide a comparison content, and
(c). an output module displaying the comparison content for the user, wherein if the measured level of xanthophyll from the subject is lower than a reference level of xanthophyll indicates that the subject affected with MCI or if there is a reduction of at least 10% to a prior reading, then the subject likely has developed MCI and is advised to take xanthophyll dietary supplement.

33. The system of claim 32, wherein the control data comprises data from a plurality of subjects who do not have symptoms associated with MCI.
